(19) United States
(12) Patent Application Publication
(10) Pub. No.: US 2023/0032826 A1
(43) Pub. Date: Feb. 2, 2023

(54) PRODUCTION OF RECOMBINANT VIRAL VECTORS FROM PLANT HAIRY ROOTS

(71) Applicant: GENETHON, EVRY (FR)

(72) Inventors: SERGE BRAUN, DORLISHEIM (FR); FLORIAN CARDON, ROIGLISE (FR); YOHANN DICKX, PARIS (FR); MARINA GUILLET, BLAIN (FR); SAMIA MARTIN, SAVIGNY SUR ORGE (FR); MARIE RANDOUX, AMIENS (FR)

(21) Appl. No.: 17/785,422

(22) PCT Filed: Dec. 18, 2020
(86) PCT No.: PCT/EP2020/086966

§ 371 Date: Jun. 15, 2022

(30) Foreign Application Priority Data
Dec. 18, 2019 (EP) 19306687.5

(51) Int. Cl. C12N 15/82; C12N 15/86

(52) U.S. Cl. CPC C12N 15/8257; C12N 15/86; C12N 15/8205; C12N 2750/14143; C12N 2750/14151

(57) ABSTRACT

The present invention relates to a method for producing a recombinant viral vector from hairy roots of a plant, in particular from hairy roots of a plant belonging to the Brassicaceae family. The invention also relates to a transgenic plant, a hairy root culture and a recombinant viral vector obtainable by the method of the invention.

### FIELD OF THE INVENTION

The present invention relates to a method for producing recombinant viral vectors from hairy roots, in particular hairy roots of a plant belonging to the Brassicaceae family.

### BACKGROUND OF THE INVENTION

Advances in the use of recombinant viral vectors for gene therapy and DNA vaccination applications have created a need for efficient production systems. Examples of recombinant viral vectors of therapeutic interest include lentivirus-based vectors and adeno-associated virus-based vectors.

Of the gene therapy products in development, recombinant Adeno-Associated Virus (AAV)-based vectors are currently the most widely used and show the greatest potential for in vivo delivery. The preferred use of rAAV vector systems is due, in part, to the lack of disease associated with the wild-type virus, the ability of AAV to transduce non-dividing as well as dividing cells, and the resulting long-term robust transgene expression observed in several Phase I/II/III trials. Furthermore, different rAAV vector serotypes can be exploited to specifically target different tissues, organs, and cells. The recent market approvals of recombinant adeno-associated virus gene therapies in Europe and the United States (such as Luxturna® or Zolgensma®) represent landmark achievements in the field of gene therapy.

AAV are non-enveloped icosahedral particles containing a single stranded DNA genome. AAV belongs to the dependoparvovirus, a genus that depends on a helper virus to provide essential genes in trans for productive infection (Weitzman and Linden, 2011). The 4.7 kb genome contains two main open reading frame, the regulatory (Rep) and structural capsid (Cap) genes, that encode for the multiple proteins required for viral replication, capsid structure, and packaging of the viral genome. Three proteins, VP1, VP2 and VP3, are naturally produced from the cap gene through a combination of alternative splicing and leaky scanning of transcripts from the p40 promoter. They all share the same C terminal sequence. AAV capsids are composed of 60 units of VP1, VP2 and VP3 in an approximate ratio of 1:1:10. A protein called Assembly-Activating protein (AAP) is translated from a different open reading frame in the cap gene and is required for capsid assembly (Sonntag et al., 2010). Four proteins are produced from the rep gene: the two largest proteins, Rep 78 and Rep 68 come from a transcription initiated using the p5 promoter while the two other proteins, Rep 52 and Rep 40, derived from a transcription using the p19 promoter. In addition to the Rep proteins, replication and encapsulation of the AAV DNA also require inverted terminal repeats (ITRs) (Balakrishnan and Jayandharan, 2014; Robert et al., 2017).

In the case of rAAVs, a number of production strategies exist to generate viral vectors.

Transient transfection of plasmid DNA into mammalian cells for the production of AAV viral vectors is the strategy most commonly used in clinical grade manufacturing of these viral vectors. rAAV vectors are usually produced in human embryonic kidney 293 cells (HEK293), following transfection of typically three DNA plasmids carrying the Rep and Cap genes, the rAAV transgene, and the specific genes that provide helper adenovirus function.

Generation of stable engineered cell lines, through the introduction of both Rep and Cap genes and/or the rAAV genome, give rise to packaging or producer cell lines.

The insect cell/baculovirus system has been developed for the production of AAVs by Urabe et al. (2006). The first generation was based on three different baculoviruses (BV) inserted into the polyhedrin locus. The three recombinant BV vectors encoded the transgenes for Rep, Cap, and rAAV genome, respectively. A second generation of BV was developed in which the number of BV was reduced to two (Smith et al., 2009). In this method, the rep and cap sequences were inserted into a single baculovirus in a head to head position.

In addition, a system for AAV production using co-expression was established in yeast by Barajas et al., 2017.

Despite the improvement of these systems within the last few years, the productivity and/or quality of the vectors have to be enhanced to allow a routine use of gene therapy.

There is thus a need for an alternative system for producing recombinant viral vectors such as rAAV vector.

### SUMMARY OF THE INVENTION

A first aspect of the invention relates to a method for producing a recombinant mammalian viral vector from hairy roots of a plant comprising the steps of:

a) inducing the formation of hairy roots from said plant;

and

b) transforming said plant with at least one vector containing one or more expression cassette(s);

wherein the one or more expression cassette(s) comprise genes encoding the protein components required for the production of the recombinant viral vector;

wherein said plant belongs to the Brassicaceae family.

In particular, said plant belonging to the Brassicaceae family may be selected from the group consisting of Raphanus sativus, Raphanus sativus var. niger, Brassica oleracea L. convar, Brassica napus, Arabidopsis thaliana and Brassica rapa, the plant being in particular Brassica rapa.

In a particular embodiment, step a) is carried out by transforming the plant with a bacterial strain comprising the rol genes, wherein the bacterial strain is able to infect the plant.

In a particular embodiment, the bacterial strain is Rhizobium rhizogenes or Agrobacterium tumefaciens.

In a particular embodiment, the recombinant viral vector is a recombinant adeno-associated virus (AAV) viral vector.

In a particular embodiment, the one or more expression cassette(s) comprise AAV rep and cap genes. In a particular embodiment, each of the AAV rep and cap genes is under the control of a promoter derived from a virus infecting Brassicaceae plants such as the cauliflower mosaic virus 35S (CaMV35S) promoter.

In another particular embodiment, the one or more expression cassette(s) comprise the genes encoding VP1, VP2, VP3, AAP (Assembly Activating Protein), Rep52 and Rep78 protein. In a particular embodiment, each gene encoding VP1, VP2, VP3, AAP, Rep52 or Rep78 protein is under the control of a constitutive promoter such as the cauliflower mosaic virus 35S (CaMV35S) promoter or the nopaline synthase (nos) promoter, or under the control of an inducible promoter such as the alcohol dehydrogenase (AlcA) promoter.

In a particular embodiment, the gene encoding VP1 is under the control of the nos promoter, the gene encoding VP2 is under the control of the nos promoter, the gene encoding VP3 is under the control of the CaMV35S promoter or a functional variant thereof, and the gene encoding AAP is under the control of the CaMV35S promoter or a functional variant thereof.

In a particular embodiment, the gene encoding VP3 is under the control of a functional variant of CaMV35S promoter having at least 80%, 85%, 90%, 95%, 99% or 100% identity to the nucleotide sequence of SEQ ID NO: 13 and the gene encoding AAP is under the control of a functional variant of CaMV35S promoter having at least 80%, 85%, 90%, 95%, 99% or 100% identity to the nucleotide sequence of SEQ ID NO: 13.

In a particular embodiment, each gene encoding VP1, VP2, VP3 and AAP is under the control of an AlcA promoter.

In a particular embodiment, the gene encoding VP3 is further under the control of an enhancer, in particular the Tobacco Mosaic Virus Omega (TMVΩ) enhancer.

In a particular embodiment, each gene encoding Rep52 and Rep78 is under the control of an AlcA promoter.

In a particular embodiment, the gene encoding Rep52 is further under the control of an enhancer, in particular the Tobacco Mosaic Virus Omega (TMVΩ) enhancer.

In a particular embodiment, the gene encoding VP1, VP2, VP3, AAP, Rep52 and/or Rep78 protein is codon optimized.

In a particular embodiment, the plant is further transformed with a vector coding for viral helper functions needed for an efficient replication of the virus, in particular a vector coding for the adenoviral helper functions.

In a particular embodiment, the plant is further transformed with a vector that comprises a viral genome comprising a gene encoding a product of interest, in particular a vector comprising a gene encoding a product of interest flanked by two AAV-ITR sequences.

Another aspect of the invention relates to a hairy root culture obtainable by:

a) inducing the formation of hairy roots from a plant;

and

b) transforming said plant with at least one vector containing one or more expression cassette(s);

wherein the one or more expression cassette(s) comprise genes encoding the protein components required for the production of the recombinant viral vector;

wherein said plant belongs to the Brassicaceae family.

In a particular embodiment, the hairy root culture is obtainable by transforming a plant belonging to the Brassicaceae family, wherein the plant is selected from the group consisting of Raphanus sativus, Raphanus sativus var. niger, Brassica oleracea L. convar, Brassica napus, Arabidopsis thaliana and Brassica rapa, the plant being in particular Brassica rapa.

In a particular embodiment, the recombinant mammalian viral vector is a recombinant adeno-associated virus (AAV) viral vector. In another particular embodiment, the one or more expression cassette(s) are as defined above.

A further aspect of the invention relates to a recombinant mammalian viral vector obtainable by the method as described above. In a particular embodiment, said recombinant mammalian viral vector is produced from hairy roots of a plant belonging to the Brassicaceae family, wherein said plant is selected from the group consisting of Raphanus sativus, Raphanus sativus var. niger, Brassica oleracea L. convar, Brassica napus, Arabidopsis thaliana and Brassica rapa, the plant being in particular Brassica rapa. In a particular embodiment, the recombinant mammalian viral vector is a recombinant adeno-associated virus (AAV) viral vector. In another particular embodiment, the recombinant mammalian viral vector is produced from hairy roots of a plant belonging to the Brassicaceae family, wherein the plant is transformed with at least one vector containing one or more expression cassette(s); wherein the one or more expression cassette(s) comprise genes encoding the protein components required for the production of the recombinant viral vector and wherein the one or more expression cassette(s) are as defined above.

Another aspect of the invention relates to a transgenic plant transformed with at least one vector containing one or more expression cassette(s); wherein the one or more expression cassette(s) comprise genes encoding the protein components required for the production of a recombinant mammalian viral vector;

and wherein said plant belongs to the Brassicaceae family.

In a particular embodiment, the transgenic plant belonging to the Brassicaceae family is selected from the group consisting of Raphanus sativus, Raphanus sativus var. niger, Brassica oleracea L. convar, Brassica napus, Arabidopsis thaliana and Brassica rapa, the plant being in particular Brassica rapa.

In a particular embodiment, the recombinant mammalian viral vector is a recombinant adeno-associated virus (AAV) viral vector. In another particular embodiment, the one or more expression cassette(s) are as defined above.

### LEGENDS TO THE FIGURES

FIG. 1: Schematic representation of constructs designed for production of AAV proteins in hairy roots.

This FIGURE shows five examples of constructs envisioned for testing the ability of Brassica rapa hairy roots to produce AAV proteins.

Construct 1 comprises: (i) a first expression cassette comprising the CaMV35S promoter (“p35S”), the Cap gene (“CAP”), and the CaMV35S terminator (“t35S”) and (ii) a second expression cassette comprising the CaMV35S promoter (“p35S”), the Rep gene (“Rep”), and the nos terminator (“tNOS”). Construct 2 comprises: (i) a first expression cassette comprising the nos promoter (“pNOS”), the VP1 gene (“VP1”), and the nos terminator (“tNOS”); (ii) a second expression cassette comprising the nos promoter (“pNOS”), the VP2 gene (“VP2”), and the nos terminator (“tNOS”); (iii) a third expression cassette comprising a variant of the CaMV35S promoter (called “p2*35S”), the VP3 gene (“VP3”), and the CaMV35S terminator (“t35S”); and (iv) a fourth expression cassette comprising a variant of the CaMV35S promoter (called “p2*35S”), the AAP gene (“AAP”), and the CaMV35S terminator (“t35S”). The “p2*35S” promoter is a variant of the CaMV35S promoter comprising a duplication of the −343 to −90 bp fragment, as described in Kay et al., 1987.

Construct 3 comprises (i) a first expression cassette comprising the inducible promoter of the Alcohol dehydrogenase (“pAlcA”), the Rep52 gene (“Rep52”) and the CaMV35S terminator (“t35S”); (ii) a second expression cassette comprising the inducible promoter of the Alcohol dehydrogenase (“pAlcA”), the Rep78 gene (“Rep78”) and the nos terminator (“tNOS”); and (iii) a third expression cassette comprising the CaMV35S promoter (“p35S”), the gene encoding ALCR protein necessary for the activation of the alcohol dehydrogenase promoter (“AlcR”), and the CaMV35S terminator (“t35S”). Construct 4 comprises (i) a first expression cassette comprising the inducible promoter of the Alcohol dehydrogenase (“pAlcA”), the Tobacco Mosaic Virus Omega enhancer (“TMVΩ”), the Rep52 gene (“Rep52”) and the CaMV35S terminator (“t35S”); (ii) a second expression cassette comprising the inducible promoter of the Alcohol dehydrogenase (“pAlcA”), the Rep78 gene (“Rep78”) and the nos terminator (“tNOS”); and (iii) a third expression cassette comprising the CaMV35S promoter (“p35S”), the gene encoding ALCR protein necessary for the activation of the alcohol dehydrogenase promoter (“AlcR”), and the CaMV35S terminator (“t35S”).

Construct 5 comprises (i) a first expression cassette comprising the inducible promoter of the Alcohol dehydrogenase (“pAlcA”), the VP1 gene (“VP1”) and the nos terminator (“tNOS”); (ii) a second expression cassette comprising the inducible promoter of the Alcohol dehydrogenase (“pAlcA”), the VP2 gene (“VP2”) and the nos terminator (“tNOS”); (iii) a third expression cassette comprising the inducible promoter of the Alcohol dehydrogenase (“pAlcA”), the Tobacco Mosaic Virus Omega enhancer (“TMVΩ”), the VP3 gene (“VP3”) and the CaMV35S terminator (“t35S”); (iv) a fourth expression cassette comprising the inducible promoter of the Alcohol dehydrogenase (“pAlcA”), the AAP gene (“AAP”) and the CaMV35S terminator (“t35S”); and (v) a fifth expression cassette comprising the CaMV35S promoter (“p35S”), the gene encoding ALCR protein necessary for the activation of the alcohol dehydrogenase promoter (“AlcR”), and the CaMV35S terminator (“t35S”).

### DETAILED DESCRIPTION OF THE INVENTION

A first aspect of the invention relates to a method for producing a recombinant mammalian viral vector from hairy roots of a plant comprising the steps of:

a) inducing the formation of hairy roots from said plant;

and

b) transforming said plant with at least one vector containing one or more expression cassette(s);

wherein the one or more expression cassette(s) comprise genes encoding the protein components required for the production of the recombinant viral vector;

wherein said plant belongs to the Brassicaceae family.

### Recombinant Mammalian Viral Vector

The term “viral vector”, in accordance with the present invention, relates to a carrier, i.e. a “vector” that is derived from a virus. This term includes any viral particle carrying or lacking a viral genome.

In the context of the present invention, a viral vector lacking a viral genome is also referred to as a virus-like particle (VLP). VLPs are highly organized structures that self-assemble from virus-derived structural proteins. These stable and versatile nano-particles possess excellent adjuvant properties capable of inducing innate and acquired immune responses. During the past years, VLPs have been applied in other branches of biotechnology taking advantage of their structural stability and tolerance towards manipulation to carry and display heterologous molecules or serve as building blocks for novel nanomaterials. VLPs can be produced from components of a wide variety of virus families including Parvoviridae (e.g. adeno-associated virus), Retroviridae (e.g. HIV), Flaviviridae (e.g. Hepatitis C virus), Paramyxoviridae (e.g. Nipah) and bacteriophages.

The term “viral vector carrying a viral genome” refers in particular to infectious viral particles. The genome of the recombinant viral vector is modified, as compared to a wild-type (wt) viral genome, by replacement of a part of the wt genome with a transgene of interest. The term “transgene of interest” refers to a gene whose nucleic acid sequence is non-naturally occurring in the viral genome. The transgene of interest may be a coding sequence or non-coding sequence. In particular, the recombinant viral vector is to be used in gene therapy. As used herein, the term “gene therapy” refers to the transfer of genetic material (e.g., DNA or RNA) of interest into a host to treat or prevent a genetic or acquired disease or condition. The genetic material of interest encodes a product (e.g., a polypeptide or functional RNA) whose production in vivo is desired. For example, the genetic material of interest can encode a hormone, receptor, enzyme or polypeptide of therapeutic value. Alternatively, the genetic material of interest can encode a functional RNA of therapeutic value, such as an antisense RNA (for example an antisense RNA suitable for exon-skipping) or a shRNA of therapeutic value.

In the context of the present invention, the viral vector is a mammalian viral vector, i.e. it is derived from a virus able to infect mammals, in particular humans. Thus, in the context of the present invention, the viral vector does not derive from a virus able to infect plants.

The recombinant viral vector may in particular derive from an adenovirus, a parvovirus (in particular an adeno-associated virus), a retrovirus (in particular a lentivirus or a spumavirus), an herpes simplex virus, an alphavirus, a flavivirus, a rhabdovirus, a measles virus, a newcastle disease virus, a picornavirus or a poxvirus.

In a particular embodiment, the recombinant viral vector is a recombinant AAV (rAAV) vector.

In the present invention, the capsid of the AAV vector may be derived from a naturally or non-naturally-occurring serotype. In a particular embodiment, the serotype of the capsid of the AAV vector is selected from AAV natural serotypes. Alternatively to using AAV natural serotypes, artificial AAV capsids may be used in the context of the present invention, including, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. A capsid from an artificial AAV serotype may be, without limitation, a chimeric AAV capsid, a recombinant AAV capsid, or a “humanized” AAV capsid.

According to a particular embodiment, the capsid of the AAV vector is of the AAV-1, -2, AAV-2 variants (such as the quadruple-mutant capsid optimized AAV-2 comprising an engineered capsid with Y44+500+730F+T491V changes, disclosed in Ling et al., 2016), -3 and AAV-3 variants (such as the AAV3-ST variant comprising an engineered AAV3 capsid with two amino acid changes, S663V+T492V, disclosed in Vercauteren et al., 2016), -3B and AAV-3B variants, -4, -5, -6 and AAV-6 variants (such as the AAV6 variant comprising the triply mutated AAV6 capsid Y731F/Y705F/T492V form disclosed in Rosario et al., 2016), -7, -8, -9 and AAV-9 variants (such as AAVhu68), -2G9, -10 such as -cy10 and -rh10, -rh39, -rh43, -rh74, -dj, Anc80, LK03, AAV.PHP, AAV2i8, porcine AAV such as AAVpo4 and AAVpo6, and tyrosine, lysine and serine capsid mutants of AAV serotypes. In addition, the capsid of other non-natural engineered variants (such as AAV-spark100), chimeric AAV or AAV serotypes obtained by shuffling, rationale design, error prone PCR, and machine learning technologies can also be useful.

In a particular embodiment, the AAV vector is a chimeric vector, i.e. its capsid comprises VP capsid proteins derived from at least two different AAV serotypes, or comprises at least one chimeric VP protein combining VP protein regions or domains derived from at least two AAV serotypes. For example a chimeric AAV vector can derive from the combination of an AAV8 capsid sequence with a sequence of an AAV serotype different from the AAV8 serotype, such as any of those specifically mentioned above. In another embodiment, the capsid of the AAV vector comprises one or more variant VP capsid proteins such as those described in WO2015013313, in particular the RHM4-1, RHM15-1, RHM15-2, RHM15-3/RHM15-5, RHM15-4 and RHM15-6 capsid variants. In a particular embodiment, the capsid of the AAV vector is a hybrid between AAV serotype 9 (AAV9) and AAV serotype 74 (AAVrh74) capsid proteins. For example, the AAV serotype may be a -rh74-9 serotype as disclosed in WO2019/193119 (such as the Hybrid Cap rh74-9 serotype described in examples of WO2019/193119; a rh74-9 serotype being also referred to herein as “-rh74-9”, “AAVrh74-9” or “AAV-rh74-9”) or a -9-rh74 serotype as disclosed in WO2019/193119 (such as the Hybrid Cap 9-rh74 serotype described in the examples of WO2019/193119; a -9-rh74 serotype being also referred to herein as “−9-rh74”, “AAV9-rh74”, “AAV-9-rh74”, or “rh74-AAV9”). In a particular embodiment, the capsid of the AAV vector is a peptide-modified hybrid between AAV serotype 9 (AAV9) and AAV serotype 74 (AAVrh74) capsid proteins, as described in PCT/EP2019/076958, such as an AAV9-rh74 hybrid capsid or AAVrh74-9 hybrid capsid modified with the P1 peptide.

In another embodiment, the modified capsid can be derived also from capsid modifications inserted by error prone PCR and/or peptide insertion (e.g. as described in Bartel et al., 2011). In addition, capsid variants may include single amino acid changes such as tyrosine mutants (e.g. as described in Zhong et al., 2008).

In a particular embodiment, the AAV vector has a naturally occurring capsid, such as an AAV1, AAV2, AAV3, AAV4, AAV5, AAV6, AAV7, AAV8, AAV9, AAV-cy10, AAVrh10 capsid. In a particular embodiment, the recombinant AAV vector has an AAV8 capsid.

The genome of the AAV vector comprises 5′- and 3′-AAV inverted terminal repeats (ITRs) optionally flanking a genetic material of interest. The ITRs may be derived from any AAV genome, such as an AAV1, AAV2, AAV3, AAV4, AAV5, AAV6, AAV7, AAV8, AAV9, AAV-cy10 or AAVrh10 genome. In a particular embodiment, the genome of the AAV vector comprises 5′- and 3′-AAV2 ITRs.

In addition, the genome of the AAV vector may either be a single stranded or self-complementary double-stranded genome (McCarty et al., Gene Therapy, 2003). Self-complementary double-stranded AAV vectors are generated by deleting the terminal resolution site (trs) from one of the AAV terminal repeats. These modified vectors, whose replicating genome is half the length of the wild type AAV genome have the tendency to package DNA dimers.

Any combination of AAV serotype capsid and ITR may be implemented in the context of the present invention, meaning that the AAV vector may comprise a capsid and ITRs derived from the same AAV serotype, or a capsid derived from a first serotype and ITRs derived from a different serotype than the first serotype. Such a vector with capsid ITRs deriving from different serotypes is also termed a “pseudotyped vector”.

### Plant Belonging to the Brassicaceae Family

As used herein, the expression “plant belonging to the Brassicaceae family” has its general meaning in the art. It encompasses any plant of the Brassicaceae family, also known as the crucifers, the mustard family or cabbage family.

It contains over 330 genera and about 3,700 species, according to the Royal Botanic Gardens, Kew. The largest genera are Draba (365 species), Cardamine (200 species), Erysimum (225 species), Lepidium (230 species) and Alyssum (195 species.)

Well-known species include, without limitation, Brassica oleracea (cabbage, cauliflower, etc.), Brassica rapa (turnip, Chinese cabbage, etc.), Brassica napus (rapeseed, etc.), Raphanus sativus (common radish), Armoracia rusticana (horseradish), Matthiola (stock), Arabidopsis thaliana (model organism) and many others. Among these species, several produce edible roots (turnip, radish . . . )

In a preferred embodiment, said plant belonging to the Brassicaceae family is selected from the group consisting of Brassica rapa, Raphanus sativus, Raphanus sativus var. niger, Brassica Oleracea L. convar, Brassica napus and Arabidopsis thaliana.

More preferably, said plant belonging to the Brassicaceae family is Brassica rapa or Brassica napus.

Even more preferably, said plant belonging to the Brassicaceae family is Brassica rapa.

Step a) of Inducing the Formation of Hairy Roots from Said Plant;

Any technique able to induce the formation of hairy roots in a plant can be used in the invention.

Hairy roots is a type of proliferating roots that emerge at the wounding site of a plant, following an infection caused by Agrobacterium rhizogenes, a gram negative soil bacterium. The hairy root phenotype is characterized by fast hormone-independent growth, lateral branching, genetic stability and lack of geotropism.

It should be noted that Agrobacterium rhizogenes has been renamed Rhizobium rhizogenes following taxonomic changes to the genus Agrobacterium and the family of the Rhizobiaceae. Rhizobium rhizogenes can also be identified by the name Agrobacterium rhizogenes.

Hairy roots was first identified as a disease in select plants caused by Rhizobium rhizogenes, which can be isolated from the soil. The gram-negative bacterium transfers DNA from its root-inducing (Ri) plasmid into the genome of the infected plant cell which results in the formation of roots. In particular, the rol genes containing genes rolA, rolB and rolC genes (F. F. White et al., 1983) are present in the T-DNA of Rhizobium rhizogenes Ri plasmid and expression of these genes induce the formation of hairy roots.

In a particular embodiment, formation of hairy roots is induced by transforming the plant with a bacterial strain comprising the rol genes, wherein the bacterial strain is able to infect the plant.

The expression “rol genes” as used herein has its general meaning in the art. It refers to the group of bacterial genes which are capable of inducing the formation of hairy roots (Schmülling et al., 1988; Bulgakov et al., 2008). Typically, the rol genes are harbored by a plasmid such as a pRi plasmid.

In a particular embodiment, the bacterial strain naturally comprises rol genes in its genome, or is modified by introduction of heterologous rol genes.

In a particular embodiment, the bacterial strain belongs to the Rhizobium genus.

In a preferred embodiment, a strain of Rhizobium rhizogenes is used.

Several strains of Rhizobium rhizogenes can be used for carrying out the invention. Suitable strains include, but are not limited to, the Rhizobium rhizogenes strain TR7, also known as ATCC 25818 and the strains LBA 9402, A4T, A4, LBA1334, ATCC 11325, ATCC 15834, LMG 155, HRI, TR105, ATCC 39207, R1000, LBA 9422, strain 1072, BL311, R1600, R1601, C58C1, A4RS, MSU440, ARqua1, 8194, TR101, 2659, LBA8490, NIAES1724, C8 (MAFF03-10268) and DC-AR2.

In a preferred embodiment, said strain of Rhizobium rhizogenes is the strain ATCC 15834 or ATCC 25818.

Even more preferably, the strain of Rhizobium rhizogenes is the strain ATCC 15834.

In another embodiment, a strain of Agrobacterium tumefaciens is used. In a particular embodiment, the strain of Agrobacterium tumefaciens that is used has been modified in order to introduce in its genome the rol genes. In a particular embodiment, Agrobacterium tumefaciens has been modified by transformation with a pRi plasmid comprising the rol genes.

In another embodiment, the strain of Agrobacterium tumefaciens that is used does not comprise the rol genes. In this embodiment, transforming the plant with Agrobacterium tumefaciens induces the formation of callus tissues. Said callus tissues are then differentiated in hairy roots following the addition of one or more hormonal substance(s). In a particular embodiment said hormonal substance is a hormone of the auxin family such as 1-Naphthaleneacetic acid (NAA), Indole-3-acetic acid (IAA) or Indole-3-butyric acid (IBA).

Several strains of Agrobacterium tumefaciens can be used for carrying out the invention. Suitable strains include, but are not limited to, A. tumefaciens C58, C58C1, LBA4404, GV2260, GV3100, A136, GV3101, GV3850, EHA101, EHA105, AGL-1.

Transformation by the bacterial strain such as Rhizobium rhizogenes and/or Agrobacterium tumefaciens is a known technique in the art. The skilled person is familiar with the different techniques commonly employed for carrying out said transformation step. According to the plant species to be transformed, different parts of the plant can be used for the infection. Such plant parts can include, for example and without limitation, seed, plant stem, leaves, petiole, cotyledonary node, hypocotyl, or other plant parts or cells.

Typically, infection by Rhizobium rhizogenes and/or Agrobacterium tumefaciens is carried out by applying a Rhizobium rhizogenes and/or Agrobacterium tumefaciens inoculum to plant tissues which has been previously wounded.

A semi-solid medium or liquid nutrient solution is preferably employed which is optimized for maintenance of hairy roots, resulting in increased growth rate and productivity of hairy roots compared to non-infected plant cells. While many types of material and solutions and medium are known and can be used in the invention, several preferred examples include Murashige and Skoog medium (MS) and Gamborg B5 medium. Several media modifications optimized for meeting nutrient requirements of the host plants used in making sustainable hairy root cultures can be employed.

In a particular embodiment, step a) of inducing the formation of hairy roots is carried out before step b) of transforming the plant with at least one vector containing one or more expression cassette(s), wherein the one or more expression cassette(s) comprise genes encoding the protein components required for the production of the recombinant viral vector.

In another particular embodiment, step a) of inducing the formation of hairy roots is carried out after step b) of transforming the plant with at least one vector containing one or more expression cassette(s), wherein the one or more expression cassette(s) comprise genes encoding the protein components required for the production of the recombinant viral vector. In this embodiment, hairy roots can be obtained by transforming a transgenic plant which expresses the genes encoding the protein components required for the production of the recombinant viral vector, with a bacterial strain comprising the rol genes and able to infect the plant as defined above.

In a preferred embodiment, steps a) and b) are performed simultaneously.

In a particular embodiment, the bacterial strain comprising the rol genes and able to infect the plant as defined above further comprises in its genome one or more expression cassette(s), wherein the one or more expression cassette(s) comprise genes encoding the protein components required for the production of the recombinant viral vector.

Thus, in this embodiment, the method for producing a recombinant mammalian viral vector from hairy roots of a plant comprises the step of transforming said plant with a bacterial strain such as Rhizobium rhizogenes or Agrobacterium tumefaciens;

wherein the bacterial strain comprises in its genome the rol genes and one or more expression cassette(s);

wherein the one or more expression cassette(s) comprise genes encoding the protein components required for the production of the recombinant viral vector;

and wherein said plant belongs to the Brassicaceae family.

In another particular embodiment, two bacterial strains are used simultaneously, wherein one bacterial strain comprises the rol genes and wherein the other bacterial strain comprises the one or more expression cassette(s) encoding the protein components required for the production of the recombinant viral vector.

In another particular embodiment, two or more bacterial strain(s) are used simultaneously, wherein the two or more bacterial strains comprise different expression cassette(s) encoding the protein components required for the production of the recombinant viral vector, and wherein at least one bacterial strain comprises the rol genes.

In a particular embodiment, three or more bacterial strains are used simultaneously, wherein one bacterial strain comprises the rol genes; and at least two or more bacterial strains comprise different expression cassette(s) encoding the protein components required for the production of the recombinant viral vector.

Step b) of Transforming the Plant with at Least One Vector Containing One or More Expression Cassette(s)

As described above, the method of the invention comprises a step b) of transforming the plant with at least one vector containing one or more expression cassette(s), wherein the one or more expression cassette(s) comprise genes encoding the protein components required for the production of the recombinant viral vector.

In a particular embodiment, the plant is further transformed with a vector coding for the viral helper functions needed for an efficient replication of the virus, such as a vector coding for the adenoviral helper functions when the recombinant viral vector is a rAAV vector.

In a particular embodiment, the plant is further transformed with a vector comprising a viral genome that comprises a gene encoding a product of interest. In a further particular embodiment, the vector comprises a gene encoding a product of interest flanked by two AAV-ITR sequences.

Any technique enabling the transformation of a plant, in particular a plant belonging to the Brassicaceae family, can be used. In particular, any physical method can be used such as gene gun method or biolistic system, electroporation, microinjection or ultrasound-mediated transformation. Chemical method can also be used such as liposome-mediated transformation, silicon carbide fibre mediated-transformation, PEG-mediated transformation, Calcium phosphate co-precipitation, polycation DMSO technique or DEAE dextran procedure. PEI-mediated transformation can also be used.

In a preferred embodiment, transformation is carried out with a bacterial strain such as Rhizobium rhizogenes or Agrobacterium tumefaciens that naturally transfers DNA (T-DNA) located on the tumor inducing (Ti) plasmid into the nucleus of plant cells and stably incorporates the DNA into the plant genome. In a particular embodiment, Rhizobium rhizogenes or Agrobacterium tumefaciens comprises a binary vector which comprise in its T-DNA region one or more expression cassette(s) as defined below. Said Rhizobium rhizogenes or Agrobacterium tumefaciens further comprises an helper plasmid containing the vir genes that originated from the Ti plasmid of Agrobacterium. These genes code for a series of proteins that cut the binary plasmid at the left and right border sequences, and facilitate transduction of the T-DNA to the host plant's cells.

In a preferred embodiment, the bacterial strain such as Rhizobium rhizogenes or Agrobacterium tumefaciens comprising the one or more expression cassette(s) encoding the protein components required for the production of the recombinant viral vector, further comprises the rol genes required for the formation of hairy roots.

In a particular embodiment, two or more bacterial strain are used, wherein the two or more bacterial strains comprises different expression cassette(s) encoding the protein components required for the production of the recombinant viral vector.

As used herein, the term “expression cassette” has its general meaning in the art. It refers to a combination of elements required for the expression of one or more genes.

In one aspect, the present invention also relates to such expression cassettes.

The expression cassette(s) can be contained in any suitable expression vector(s). Typically, the expression vector may be a binary vector suitable for expression in a plant cell, such as the pRD400, pBIN19, pBINPlus or pCAMBIA binary vector, which has been modified to include the one or more expression cassette(s) of the invention. Other examples of binary vectors are described in Table 2 of Bahramnejad et al., 2019. In a particular embodiment, the binary vector is pRD400.

In one embodiment, said expression cassette comprises a promoter, one or more gene(s) encoding the protein components required for the production of the recombinant viral vector, and a polyadenylation sequence. In a particular embodiment, the expression cassette further comprises an enhancer.

Any promoter suitable for expression in a plant cell may be used. In a particular embodiment, the promoter is a promoter suitable for expression in an eukaryotic cell, such as an insect or mammalian cell, and also suitable for expression in a plant cell. One skilled in the art is able to determine whether an eukaryotic promoter is able to drive the expression of a gene in a plant cell, based on his general knowledge in molecular biology. For example, a reported gene may be used for testing the ability of a promoter to which it is operatively linked in a construct, by transfecting a plant cell with a construct comprising said reporter gene and said promoter. In a particular embodiment, the promoter may be the p19 AAV promoter.

In a particular embodiment, the gene encoding the protein component required for the production of the recombinant viral vector is under the control of a constitutive promoter suitable for expression in a plant cell. A non-exhaustive list of constitutive promoters suitable for expression in a plant cell includes the promoters of the cauliflower mosaic virus 35S (CaMV35S) (Odell et al., 1985), Cassava vein mosaic virus (CVMV) (Verdaguer et al., 1996), C1 of cotton leaf curl Multan virus (CLCuMV) (Xie et al., 2003), Component 8 of milk vetch dwarf virus (Shirasawa-Seo et al., 2005), Australian banana streak virus (BSV) (Schenk et al., 2001), Mirabilis mosaic Virus (MMV) (Dey and Maiti, 1999), Figwort mosaic virus (FMV) (Sanger et al., 1990), Maize Polyubiquitin-1 (Christensen et al., 1992), Rice actin (McElroy et al., 1990), actin from Arabidopsis thaliana (An et al., 1996), nopaline synthase (nos) from Agrobacterium (An et al., 1988), rolD from Agrobacterium (Fei et al., 2003), or any functional variants thereof.

In another particular embodiment, the gene encoding the protein component required for the production of the recombinant viral vector is under the control of an inducible promoter suitable for expression in a plant cell. A non-exhaustive list of promoters suitable for expression in a plant cell includes: Pristinamycin-responsive (Frey et al., 2001), Int-2 from maize (De Veylder et al., 1997), wun1 from Potato (Siebertz et al., 1989), Mannopine synthase from Agrobacteria (Langridge et al., 1989), heat-shock promoter Gmshp17.3 from Soybean (Schöffl et al., 1989), Alcohol dehydrogenase (Felenbok et al., 1988), or any functional variants thereof.

In a particular embodiment, the inducible promoter is an Alcohol dehydrogenase promoter (Felenbok et al., 1988) or any functional variants thereof.

In a particular embodiment, the gene encoding the protein component required for the production of the recombinant viral vector is under the control of a promoter derived from a virus infecting Brassicaceae plants such as the cauliflower mosaic virus 35S (CaMV35S) promoter, or a functional variant thereof. In a particular embodiment, the promoter is a functional variant of the CaMV35S promoter, having an enhanced transcriptional activity as compared to the wild-type CaMV35S promoter. In particular, the functional variant of CaMV35S comprises a duplication of the −343 to −90 bp fragment, as described in Kay et al., 1987. In a particular embodiment, the CaMV35S functional variant has at least 80%, 85%, 90%, 95%, 99% or 100% identity to the nucleotide sequence of SEQ ID NO: 13.

By “functional variants thereof” is meant any variant keeping the functionality of the promoter from which it derives, i.e. is able to initiate the transcription of a particular gene. In particular, the functional variant may have a transcription-inducing activity of at least 50%, 60%, 70%, 80%, 90%, 95%, 99%, or at least 100% as compared to the wild-type promoter. The transcription-inducing activity of the functional variant may even be of more than 100%, such as of more than 110%, 120%, 130%, 140%, or even more than 150% of the activity of the wild-type promoter.

In a particular embodiment, the promoter is a CaMV35S promoter, such as a CaMV35S of sequence SEQ ID NO:9 or a functional variant thereof. The CAMV35S functional variant may have a transcription-inducing activity of at least 50%, 60%, 70%, 80%, 90%, 95%, 99%, or at least 100% as compared to the wild-type CAMV25S promoter of SEQ ID NO:9. The transcription-inducing activity of the functional variant may even be of more than 100%, such as of more than 110%, 120%, 130%, 140%, or even more than 150% of the activity of the wild-type CAMV25S promoter of SEQ ID NO: 9.

In a particular embodiment, the promoter is a nopaline synthase (nos) promoter, such as a nos promoter of sequence SEQ ID NO:11 or a functional variant thereof. The nos functional variant may have a transcription-inducing activity of at least 50%, 60%, 70%, 80%, 90%, 95%, 99%, or at least 100% as compared to the wild-type nos promoter of SEQ ID NO:11. The transcription-inducing activity of the functional variant may even be of more than 100%, such as of more than 110%, 120%, 130%, 140%, or even more than 150% of the activity of the wild-type nos promoter of SEQ ID NO:11.

In a particular embodiment, the promoter is an alcohol dehydrogenase promoter, such as an alcohol dehydrogenase promoter of sequence SEQ ID NO:14 or a functional variant thereof. The alcohol dehydrogenase promoter functional variant may have a transcription-inducing activity of at least 50%, 60%, 70%, 80%, 90%, 95%, 99%, or at least 100% as compared to the wild-type alcohol dehydrogenase promoter of SEQ ID NO:14. The transcription-inducing activity of the functional variant may even be of more than 100%, such as of more than 110%, 120%, 130%, 140%, or even more than 150% of the activity of the wild-type alcohol dehydrogenase promoter of SEQ ID NO:14.

In a particular embodiment, the expression cassette comprises a terminator sequence downstream of the gene encoding the protein component required for the production of the recombinant viral vector. By “terminator sequence” is meant a sequence comprising 3′ UTR sequences and polyadenylation signal to terminate the transcription. Any terminator sequence able to mediate transcriptional termination in a plant cell, may be used.

In a particular embodiment, the terminator sequence is the nopaline synthase (nos) terminator or the CAMV35S terminator.

The genes encoding the protein components required for the production of the recombinant viral vector are well known to those skilled in the art. Skilled persons are able to adapt the expression cassettes used in the experimental part of the description, to the particular viral vector that they wish to produce.

The genes encoding the protein components required for the production of the recombinant viral vector can be codon optimized for improving their expression in the plant cell.

The genes encoding the protein components required for the production of the recombinant viral vector comprise a start codon. For example, the start codon may be the ATG trinucleotide or alternative start codons such as ACG, CTG, GTG and TTG start codon.

In a particular embodiment, the one or more expression cassette(s) as described above comprise genes encoding the protein components required for the production of a recombinant AAV viral vector.

In a particular embodiment, the one or more expression cassette(s) of the invention comprise AAV Cap and Rep genes.

In a particular embodiment, the one or more expression cassette(s) of the invention comprises the genes encoding the AAV VP1 protein, VP2 protein and/or VP3 protein, corresponding to the structural capsid (Cap) proteins of the rAAV vector. In a particular embodiment, the one or more expression cassette(s) of the invention further comprises the gene encoding AAP protein required for capsid assembly.

In a particular embodiment, the one or more expression cassette(s) of the invention comprises the genes encoding the Rep52 protein and/or Rep78, corresponding to proteins required for viral replication.

In a particular embodiment, the AAV Cap and Rep genes are comprises in a same expression cassette. In a particular embodiment, a first expression cassette comprises the AAV Cap gene and a second expression cassette comprises the AAV Rep genes. Said first expression cassette comprising the AAV Cap gene and second expression cassette comprising the AAV Rep gene may comprise any promoter sequence suitable for expression in a plant cell, as described above. In a particular embodiment, AAV Cap gene and/or AAV Rep gene are under the control of a constitutive promoter suitable for expression in a plant cell. In another particular embodiment, AAV Cap gene and/or AAV Rep gene are under the control of an inducible promoter suitable for expression in a plant cell.

In a particular embodiment, each of the AAV Cap and Rep genes is under the control of a promoter derived from a virus infecting Brassicaceae plants such as the cauliflower mosaic virus 35S (CaMV35S) promoter. In this embodiment, a first expression cassette comprises the AAV Cap gene under the control of a CaMV35S promoter and a second expression cassette comprises the AAV Rep gene under the control of a CaMV35S promoter.

In a particular embodiment, the first expression cassette and the second expression cassette comprising respectively AAV Cap gene and AAV Rep genes further comprise a terminator sequence, as described above. Any terminator sequence able to mediate transcriptional termination in a plant cell, may be used.

In a particular embodiment, the first expression cassette comprising AAV Cap gene comprises a CaMV35S terminator sequence. In a particular embodiment, the second expression cassette comprising AAV Rep gene comprises a nopaline synthase (nos) terminator sequence.

In a particular embodiment, the first expression cassette comprises a CaMV35S promoter, an AAV Cap gene and a CaMV35S terminator sequence.

In a particular embodiment, the second expression cassette comprises a CaMV35S promoter, an AAV Rep gene and a nos terminator sequence.

In a particular embodiment, the first expression cassette and the second expression cassette further comprise an enhancer, such as an enhancer derived from tobacco mosaic virus, such as the tobacco mosaic virus omega (TMVΩ) enhancer.

In a particular embodiment, the first expression cassette and the second expression cassette are cloned into one or two expression vectors. In a preferred embodiment, the first expression cassette and the second expression cassette are cloned into a single expression vector. In a particular embodiment, the first expression cassette and the second expression cassette are cloned into a binary vector, such as the pRD400 binary vector.

The Cap and Rep genes of any AAV serotype can be used. The Cap and Rep genes can be natural or artificial sequences.

In the present invention, the Cap gene or the genes encoding VP1, VP2 or VP3 capsid proteins of the AAV vector may be derived from a naturally or non-naturally-occurring serotype. In a particular embodiment, the serotype of the capsid of the AAV vector is selected from AAV natural serotypes. Alternatively to using AAV natural serotypes, artificial AAV serotypes may be used in the context of the present invention, including, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. A capsid from an artificial AAV serotype may be, without limitation, a chimeric AAV capsid, a recombinant AAV capsid, or a “humanized” AAV capsid.

According to a particular embodiment, the Cap gene or the genes encoding VP1, VP2 or VP3 encodes a capsid of the AAV-1, -2, AAV-2 variants (such as the quadruple-mutant capsid optimized AAV-2 comprising an engineered capsid with Y44+500+730F+T491V changes, disclosed in Ling et al., 2016, -3 and AAV-3 variants (such as the AAV3-ST variant comprising an engineered AAV3 capsid with two amino acid changes, S663V+T492V, disclosed in Vercauteren et al., 2016, -3B and AAV-3B variants, -4, -5, -6 and AAV-6 variants (such as the AAV6 variant comprising the triply mutated AAV6 capsid Y731F/Y705F/T492V form disclosed in Rosario et al., 2016), -7, -8, -9 and AAV-9 variants (such as AAVhu68), -2G9, -10 such as -cy10 and -rh10, -rh39, -rh43, -rh74, -dj, Anc80, LK03, AAV.PHP, AAV2i8, porcine AAV such as AAVpo4 and AAVpo6, and tyrosine, lysine and serine capsid mutants of AAV serotypes. In addition, the Cap gene may encode a capsid of other non-natural engineered variants (such as AAV-spark100), chimeric AAV or AAV serotypes obtained by shuffling, rationale design, error prone PCR, and machine learning technologies. In a particular embodiment, the Cap gene encodes VP capsid proteins derived from at least two different AAV serotypes, or encodes at least one chimeric VP protein combining VP protein regions or domains derived from at least two AAV serotypes. For example a chimeric AAV capsid can derive from the combination of an AAV8 capsid sequence with a sequence of an AAV serotype different from the AAV8 serotype, such as any of those specifically mentioned above. In another embodiment, the capsid of the AAV vector comprises one or more variant VP capsid proteins such as those described in WO2015013313, in particular the RHM4-1, RHM15-1, RHM15-2, RHM15-3/RHM15-5, RHM15-4 and RHM15-6 capsid variants. In a particular embodiment, the capsid of the AAV vector is a hybrid between AAV serotype 9 (AAV9) and AAV serotype 74 (AAVrh74) capsid proteins. For example, the AAV serotype may be a -rh74-9 serotype as disclosed in WO2019/193119 (such as the Hybrid Cap rh74-9 serotype described in examples of WO2019/193119; a rh74-9 serotype being also referred to herein as “-rh74-9”, “AAVrh74-9” or “AAV-rh74-9”) or a -9-rh74 serotype as disclosed in WO2019/193119 (such as the Hybrid Cap 9-rh74 serotype described in the examples of WO2019/193119; a -9-rh74 serotype being also referred to herein as “−9-rh74”, “AAV9-rh74”, “AAV-9-rh74”, or “rh74-AAV9”). In a particular embodiment, the capsid of the AAV vector is a peptide-modified hybrid between AAV serotype 9 (AAV9) and AAV serotype 74 (AAVrh74) capsid proteins, as described in PCT/EP2019/076958, such as an AAV9-rh74 hybrid capsid or AAVrh74-9 hybrid capsid modified with the P1 peptide.

In a particular embodiment, the Cap gene or the genes encoding VP1, VP2 or V3 encodes a naturally occurring capsid, such as an AAV1, AAV2, AAV3, AAV4, AAV5, AAV6, AAV7, AAV8, AAV9, AAV-cy10, AAVrh10 capsid. In a particular embodiment, the Cap gene or the genes encoding VP1, VP2 or VP3 used in the present invention encodes an AAV8 capsid.

In a particular embodiment, the Rep gene of any natural AAV vector may be used. In particular, the Rep gene of AAV2 vector is used. In a particular embodiment the genes encoding Rep52 and Rep78 of AAV2 are used.

In a particular embodiment, the Cap gene comprised in the expression cassette encodes functional capsid proteins, said gene having at least 60%, 65%, 70%, 75%, 80%, 85%, 90%, 95%, 99%, 99.8% or 100% identity to the nucleotide sequence of SEQ ID NO: 1.

In a particular embodiment, the Rep gene comprised in the expression cassette encodes functional Rep proteins, said gene having at least 60%, 65%, 70%, 75%, 80%, 85%, 90%, 95%, 99%, 99.8% or 100% identity to the nucleotide sequence of SEQ ID NO: 2.

In a particular embodiment, the gene encoding VP1 protein, comprised in the expression cassette encodes a functional VP1 protein, said gene having at least 60%, 65%, 70%, 75%, 80%, 85%, 90%, 95%, 99%, 99.8% or 100% identity to the nucleotide sequence of SEQ ID NO: 3.

In a particular embodiment, the gene encoding VP1 protein, comprised in the expression cassette encodes a functional VP1 protein having at least 60%, 65%, 70%, 75%, 80%, 85%, 90%, 95%, 99%, 99.8% or 100% identity to the amino acid sequence of SEQ ID NO: 17.

In a particular embodiment, the gene encoding VP2 protein, comprised in the expression cassette encodes a functional VP2 protein, said gene having at least 60%, 65%, 70%, 75%, 80%, 85%, 90%, 95%, 99%, 99.8% or 100% identity to the nucleotide sequence of SEQ ID NO: 4.

In a particular embodiment, the gene encoding VP2 protein, comprised in the expression cassette encodes a functional VP2 protein having at least 60%, 65%, 70%, 75%, 80%, 85%, 90%, 95%, 99%, 99.8% or 100% identity to the amino acid sequence of SEQ ID NO: 18.

In a particular embodiment, the gene encoding VP3 protein, comprised in the expression cassette encodes a functional VP3 protein, said gene having at least 60%, 65%, 70%, 75%, 80%, 85%, 90%, 95%, 99%, 99.8% or 100% identity to the nucleotide sequence of SEQ ID NO: 5.

In a particular embodiment, the gene encoding VP3 protein, comprised in the expression cassette encodes a functional VP3 protein having at least 60%, 65%, 70%, 75%, 80%, 85%, 90%, 95%, 99%, 99.8% or 100% identity to the amino acid sequence of SEQ ID NO: 19.

In a particular embodiment, the gene encoding AAP protein, comprised in the expression cassette encodes a functional AAP protein, said gene having at least 60%, 65%, 70%, 75%, 80%, 85%, 90%, 95%, 99%, 99.8% or 100% identity to the nucleotide sequence of SEQ ID NO: 6.

In a particular embodiment, the gene encoding AAP protein, comprised in the expression cassette encodes a functional AAP protein having at least 60%, 65%, 70%, 75%, 80%, 85%, 90%, 95%, 99%, 99.8% or 100% identity to the amino acid sequence of SEQ ID NO: 20.

In a particular embodiment, the gene encoding Rep52 protein, comprised in the expression cassette encodes a functional Rep52 protein, said gene having at least 60%, 65%, 70%, 75%, 80%, 85%, 90%, 95%, 99%, 99.8% or 100% identity to the nucleotide sequence of SEQ ID NO: 7.

In a particular embodiment, the gene encoding Rep52 protein, comprised in the expression cassette encodes a functional Rep52 protein having at least 60%, 65%, 70%, 75%, 80%, 85%, 90%, 95%, 99%, 99.8% or 100% identity to the amino acid sequence of SEQ ID NO: 21.

In a particular embodiment, the gene encoding Rep78 protein, comprised in the expression cassette encodes a functional Rep78 protein, said gene having at least 60%, 65%, 70%, 75%, 80%, 85%, 90%, 95%, 99%, 99.8% or 100% identity to the nucleotide sequence of SEQ ID NO: 8.

In a particular embodiment, the gene encoding Rep78 protein, comprised in the expression cassette encodes a functional Rep78 protein having at least 60%, 65%, 70%, 75%, 80%, 85%, 90%, 95%, 99%, 99.8% or 100% identity to the amino acid sequence of SEQ ID NO: 22.

In a particular embodiment, the one or more expression cassette(s) of the invention comprise the genes encoding VP1 protein, VP2 protein, VP3 protein, AAP (Assembly Activating Protein), Rep52 protein and/or Rep78 protein.

The genes encoding VP1 protein, VP2 protein, VP3 protein, AAP (Assembly Activating Protein), Rep52 protein and/or Rep78 protein can be codon optimized for improving their expression in the plant cell.

The genes encoding VP1 protein, VP2 protein, VP3 protein, AAP (Assembly Activating Protein), Rep52 protein and/or Rep78 protein can be under the control of any promoter(s) enabling their expression in a plant cell.

In a particular embodiment, at least two of the genes encoding VP1, VP2, VP3, AAP, Rep52 and Rep78 are under the control of the same promoter.

In yet another embodiment, the gene encoding VP1 is comprised in an expression cassette comprising a promoter operatively linked to said gene encoding VP1; the gene encoding VP2 is comprised in an expression cassette comprising a promoter operatively linked to said gene encoding VP2; the gene encoding VP3 is comprised in an expression cassette comprising a promoter operatively linked to said gene encoding VP3; the gene encoding AAP is comprised in an expression cassette comprising a promoter operatively linked to said gene encoding AAP; the gene encoding Rep52 is comprised in an expression cassette comprising a promoter operatively linked to said gene encoding Rep52; and the gene encoding Rep78 is comprised in an expression cassette comprising a promoter operatively linked to said gene encoding Rep78.

In a particular embodiment, each cassette, comprising one gene selected from the gene encoding VP1, VP2, VP3, AAP, Rep52 or Rep78 protein is cloned into one or more vector(s). Preferably, all the cassettes, comprising each one gene selected from the gene encoding VP1, VP2, VP3, AAP, Rep52 or Rep78 protein are cloned into a same vector. In a particular embodiment, all the cassette are cloned into a binary vector, such as the pRD400 binary vector.

In a particular embodiment, the gene encoding VP1, VP2, VP3, AAP, Rep52 or Rep78 protein is under the control of a constitutive promoter suitable for expression in a plant cell. A non-exhaustive list of constitutive promoters suitable for expression in a plant cell includes the promoters of the cauliflower mosaic virus 35S (CaMV35S) (Odell et al., 1985), Cassava vein mosaic virus (CVMV) (Verdaguer et al., 1996), C1 of cotton leaf curl Multan virus (CLCuMV) (Xie et al., 2003), Component 8 of milk vetch dwarf virus (Shirasawa-Seo et al., 2005), Australian banana streak virus (BSV) (Schenk et al., 2001), Mirabilis mosaic Virus (MMV) (Dey and Maiti, 1999), Figwort mosaic virus (FMV) (Sanger et al., 1990), Maize Polyubiquitin-1 (Christensen et al., 1992), Rice actin (McElroy et al., 1990), actin from Arabidopsis thaliana (An et al., 1996), nopaline synthase (nos) from Agrobacterium (An et al., 1988), rolD from Agrobacterium (Fei et al., 2003), or any functional variants thereof.

In another particular embodiment, the gene encoding VP1, VP2, VP3, AAP, Rep52 or Rep78 protein is under the control of an inducible promoter suitable for expression in a plant cell. A non-exhaustive list of promoters suitable for expression in a plant cell includes: Pristinamycin-responsive (Frey et al., 2001), Int-2 from maize (De Veylder et al., 1997), wun1 from Potato (Siebertz et al., 1989), Mannopine synthase from Agrobacteria (Langridge et al., 1989), heat-shock promoter Gmshp17.3 from Soybean (Schöffl et al., 1989), Alcohol dehydrogenase (Felenbok et al., 1988), or any functional variants thereof.

By “functional variants thereof” is meant any variant keeping the functionality of the promoter from which it derives, i.e. is able to initiate the transcription of a particular gene.

In a particular embodiment, the gene encoding VP1, VP2, VP3, AAP, Rep52 or Rep78 protein is under the control of a promoter derived from a virus infecting Brassicaceae plants such as the cauliflower mosaic virus 35S (CaMV35S) promoter, or a functional variant thereof. In a particular embodiment, the promoter is a functional variant of the CaMV35S promoter, having an enhanced transcriptional activity as compared to the wild-type CaMV35S promoter. In particular, the functional variant of CaMV35S comprises a duplication of the −343 to −90 bp fragment, as described in Kay et al., 1987. In a particular embodiment, the CaMV35S functional variant has at least 80%, 85%, 90%, 95%, 99% or 100% identity to the nucleotide sequence of SEQ ID NO: 13.

In a particular embodiment:
The gene encoding VP1 is under the control of a weak promoter, such as the nopaline synthase (nos) promoter or a functional variant thereof;
The gene encoding VP2 is under the control of a weak promoter, such as the nopaline synthase (nos) promoter or a functional variant thereof;
The gene encoding VP3 is under the control of a strong promoter, such as the CaMV35S promoter or a functional variant thereof, preferably a functional variant having at least 80%, 85%, 90%, 95%, 99% or 100% identity to the nucleotide sequence of SEQ ID NO: 13; and/or
The gene encoding AAP is under the control of a strong promoter, such as the CaMV35S promoter or a functional variant thereof, preferably a functional variant having at least 80%, 85%, 90%, 95%, 99% or 100% identity to the nucleotide sequence of SEQ ID NO: 13.

In a particular embodiment, the at least one vector according to the invention comprises:
An expression cassette comprising the nos promoter or a functional variant thereof, a gene encoding VP1, and a terminator sequence such as a nos terminator sequence;
An expression cassette comprising the nos promoter or a functional variant thereof, a gene encoding VP2, and a terminator sequence such as a nos terminator sequence;
An expression cassette comprising the CaMV35S promoter or a functional variant thereof preferably a functional variant having at least 80%, 85%, 90%, 95%, 99% or 100% identity to the nucleotide sequence of SEQ ID NO: 13, a gene encoding VP3, and a terminator sequence such as a CamV35S terminator sequence; and/or
An expression cassette comprising the CaMV35S promoter or a functional variant thereof preferably a functional variant having at least 80%, 85%, 90%, 95%, 99% or 100% identity to the nucleotide sequence of SEQ ID NO: 13, a gene encoding AAP, and a terminator sequence such as a CamV35S terminator sequence.

In another particular embodiment:
The gene encoding VP1 is under the control of an inducible promoter, such as the alcohol dehydrogenase promoter or a functional variant thereof;
The gene encoding VP2 is under the control of an inducible promoter, such as the alcohol dehydrogenase promoter or a functional variant thereof;
The gene encoding VP3 is under the control of an inducible promoter, such as the alcohol dehydrogenase promoter or a functional variant thereof; and/or
The gene encoding AAP is under the control of an inducible promoter, such as the alcohol dehydrogenase promoter or a functional variant thereof.

In a particular embodiment, the at least one vector according to the invention comprises:
An expression cassette comprising the alcohol dehydrogenase promoter or a functional variant thereof, a gene encoding VP1, and a terminator sequence such as a nos terminator sequence;
An expression cassette comprising the alcohol dehydrogenase promoter or a functional variant thereof, a gene encoding VP2, and a terminator sequence such as a nos terminator sequence;
An expression cassette comprising the alcohol dehydrogenase promoter, a gene encoding VP3, a terminator sequence such as a CamV35S terminator sequence, and optionally an enhancer sequence such as the TMVΩ enhancer; and/or
An expression cassette comprising the alcohol dehydrogenase promoter, a gene encoding AAP, a terminator sequence such as a CamV35S terminator sequence.

In a particular embodiment, the TMVΩ enhancer has a sequence having at least 80%, 85%, 90%, 95%, 99% or 100% identity to the nucleotide sequence of SEQ ID NO: 16.

In a particular embodiment, the gene encoding VP1, VP2, VP3 and/or AAP are further codon optimized to improve their expression into the plant cell.

When the inducible promoter of the alcohol dehydrogenase is used, the at least one vector according to the invention further comprises an expression cassette encoding the ALCR protein necessary for the activation of the alcohol dehydrogenase promoter. In a particular embodiment, the gene encoding the ALCR protein encodes a functional ALCR protein, said gene having at least 60%, 65%, 70%, 75%, 80%, 85%, 90%, 95%, 99% or 100% identity to the nucleotide sequence of SEQ ID NO: 15. In a particular embodiment, the gene encoding ALCR protein, comprised in the expression cassette encodes a functional ALCR protein having at least 60%, 65%, 70%, 75%, 80%, 85%, 90%, 95%, 99%, 99.8% or 100% identity to the amino acid sequence of SEQ ID NO: 23.

In a particular embodiment:
The gene encoding Rep52 is under the control of an inducible promoter, such as the alcohol dehydrogenase promoter or a functional variant thereof; and/or
The gene encoding Rep78 is under the control of an inducible promoter, such as the alcohol dehydrogenase promoter or a functional variant thereof.

In a particular embodiment, the at least one vector according to the invention comprises:
An expression cassette comprising the alcohol dehydrogenase promoter or a functional variant thereof, a gene encoding Rep52, a terminator sequence such as a CamV35S terminator sequence, and optionally an enhancer such as the TMVΩ enhancer;

and/or
An expression cassette comprising the alcohol dehydrogenase promoter or a functional variant thereof, a gene encoding Rep78, and a terminator sequence such as a nos terminator sequence

In a particular embodiment, the gene encoding Rep52 and/or Rep78 are further codon optimized to improve their expression into the plant cell.

When the inducible promoter of the alcohol dehydrogenase is used, the at least one vector according to the invention further comprises an expression cassette encoding the ALCR protein necessary for the activation of the alcohol dehydrogenase promoter.

### Recovery of the Recombinant Viral Vector

In one embodiment, the recombinant viral vector is recovered by harvesting the medium in which the hairy roots are cultured.

In a particular embodiment, the culture medium containing said recombinant is collected and is directly used for future applications.

Advantageously, the culture medium containing the recombinant viral vector according to this embodiment is suitable for oral use in humans and/or animals without any purification step.

Thus, another aspect of the invention relates to a culture medium containing a recombinant viral vector obtainable by the method described above.

In an alternative embodiment, the recombinant viral vector is obtained after one or several purification steps. Suitable protocols adapted to each recombinant viral vector are standard techniques in the art, and the skilled person will readily select the appropriate purification step(s), if any, for the desired application.

In a second embodiment, the recombinant viral vector is extracted from the hairy roots. In particular, the recovery of the viral vector may be done by chemical extraction or by grinding the hairy roots.

In a further particular embodiment, the method for producing a recombinant mammalian viral vector further comprises a step of inducing lateral root emergences on the hairy roots of the transformed plant, as described in WO16185122.

In particular, the step of inducing lateral root emergences on the hairy roots of the transformed plant may be carried out by culturing the transformed hairy roots in the presence of at least one auxin.

In a particular embodiment, the hairy roots are cultured in a liquid medium comprising at least one auxin.

Auxin can be selected from: 2,4-dichlorophenoxyacetic acid (2,4-D), 3-indoleacetic acid (IAA), indole-3-butyric acid (IBA), 1-naphthaleneacetic acid (NAA), 2,4,5-trichlorophenoxyacetic acid (2,4,5-T), 2,3,5-triiodoacetic acid, 4-chlorophenoxyacetic acid, 2-naphthoxyacetic acid, 1-naphthylacetic acid, 4-amino-3,5,6-trichloropicolinic acid, 3,6-dichloro-2-methoxybenzoic acid (Dicamba) and derivatives thereof.

Another aspect of the invention relates to a hairy root culture obtainable, or obtained, by:

a) inducing the formation of hairy roots from a plant according to any embodiment detailed above;

and

b) transforming said plant with at least one vector containing one or more expression cassette(s) according to any embodiment detailed above; wherein the one or more expression cassette(s) comprise genes encoding the protein components required for the production of the recombinant viral vector; wherein said plant belongs to the Brassicaceae family.

Another aspect of the invention relates to a transgenic plant transformed with at least one vector containing one or more expression cassette(s); wherein the one or more expression cassette(s) comprise genes encoding the protein components required for the production of the recombinant viral vector; wherein said plant belongs to the Brassicaceae family.

Another aspect of the invention relates to a recombinant viral vector obtainable or obtained by the method as described above.

### Examples

### Materials and Methods

A. Recombinant Binary Plasmid for AAV Protein Expression

The sequences used were extracted from the plasmid p5e18_VD2_8_kanR (RepCap_cellules_293, provided by GENETHON). The coding sequences were manually optimized based on the codon usage frequency in Human (origin organism) and in Brassica rapa (expression host organism) species.

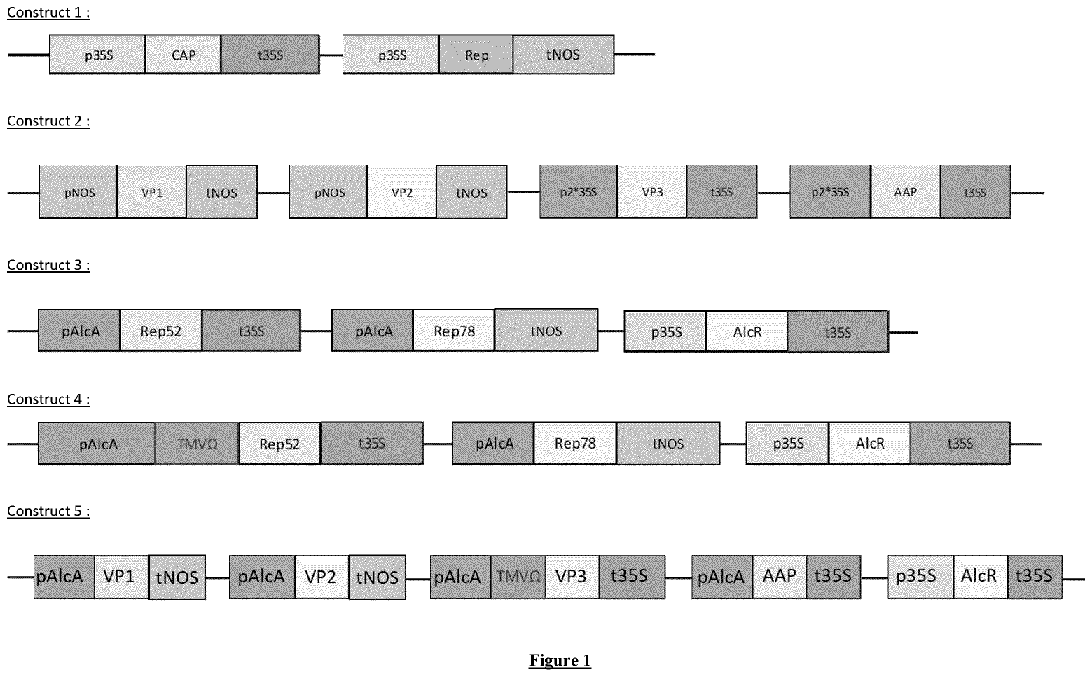

Five constructs comprising different expression cassettes were designed. Schematic representation of the five constructs is shown in FIG. 1.

In construct 1, the expression of CAP (from AAV8) and REP (from AAV2) genes is driven by the CaMV35S promoter.

In construct 2, VP1 and VP2 genes are each under the control of a Nos promoter. VP3 and AAP genes are each under the control of the “2*CaMV35S” promoter. “2*CaMV35S” promoter is a variant of CaMV35S comprising a duplication of the −343 to −90 bp fragment, as described in Kay et al., 1987. A HA tag was added to the AAP protein for enabling its detection due to the lack of specific anti-AAP antibody.

In construct 3, Rep52 and Rep78 are under the control of an ethanol inducible system.

Construct 4 is similar to the construct 3 but the Tobacco Mosaic Virus Omega (TMVΩ) enhancer was added to enhance the expression of Rep52.

In construct 5, the ethanol inducible system was used to drive the expression of VP1, VP2, VP3 and AAP. The Tobacco Mosaic Virus Omega (TMVΩ) enhancer was added to increase the expression of VP3. A HA tag was added to the AAP protein for enabling its detection due to the lack of specific anti-AAP antibody.

Each of these five constructs was then cloned, separately, in the pRD400 plant expression vector (Datla et al., 1992).

The resulting plasmid(s), pRD400-AAV was first cloned in an electrocompetent Escherichia coli strain JM101, then in Rhizobium rhizogenes strain ATCC 15834.

R. rhizogenes strain ATCC 15834 was transformed by electroporation with the AAV encoding pRD400 plasmid(s) (pRD400-AAV).

B. Production, Selection and Culture of Transgenic Hairy Roots

1. Plant Species and Culture In Vitro

Seeds from Brassica rapa rapa cv ‘Navet des vertus Marteau” were surface sterilized with 5% bleach supplemented with drops of Triton X100 for 10 min and washed at least 5 times with sterile water before to be placed on petri dishes with B5 agar. Germination and seedling growth occurred at 20° C. with a 16 h light/8 h dark photoperiod.

2. Plant Infection by Rhizobium rhizogenes

We used the Rhizobium rhizogenes strain 15834 (ATCC 15834) provided by the Institut Pasteur. Rhizobium rhizogenes was grown on MGL plates (2.5 g/l yeast extract, 5 g/l tryptone, 5 g/l mannitol, 5 g/l NaCl, 1.16 g/l Na-glutamate, 0.25 g/l KH2PO4, 0.1 g/l MgSO4, 1.0 mg/l biotin, 8 g/l Agar, pH 7.0) eventually supplemented with 50 mg/l kanamycin for selection of the binary plasmid. Inoculums were prepared from a 20 ml liquid bacterial culture grown overnight at 25° C. in MGL medium. The suspension was centrifuged for 5 min at 15,000 rpm and collected cells were resuspended in fresh MGL medium and diluted to obtain an optical density of 1±0.1 at 600 nm.

Plant infection was performed by pricking with a needle the hypocotyls of 3-10 day old seedlings and by applying the Rhizobium inoculum with a sterile cotton swab on the injured zone. Hairy roots usually emerged from the wounded site about 2 weeks after infection.

3. Selection and Culture of AAV Protein-Expressing Hairy Root Clones

Infected hypocotyls developing hairy roots were cut out from the seedling and placed on B5 (Duchefa) pH 5.8 supplemented with 3% saccharose and cefotaxime (300 mg/L), 8 g/L agar. After 7 days, independent hairy root tips were transferred on fresh B5- 3% saccharose+cefotaxime (300 mg/L), 8 g/L agar where they grow for 4 to 10 days.

To initiate cultures in liquid medium, each hairy root clone was then transferred in 6 ml of B5 with 3% of saccharose, for 10 days. This was followed by a culture medium renewal, the culture medium further comprising 2,4 dichlorophenoxyacetic acid (2,4D). Hairy root clones were cultivated for another period of 10 days. After the 20 days of culture, all samples were collected. 100 mg of fresh biomass were used for protein extraction and RNA extraction.

C. SDS-PAGE and WESTERN BLOT

Protein extraction from hairy roots was carried out using a commercial kit (Macherey-Nagel, ref 740933) and following the supplier protocol. Aliquots of hairy root extracts were sampled, mixed with ⅓ volume of 3× Laemmli buffer and boiled for 5 min. 40 μl of each sample were loaded AnykD mini protean TGX polyacrylamide gels (Bio-Rad). For Western blot analysis, proteins were transferred to nitrocellulose membranes (Bio-Rad, Hercules, Calif.) using the Bio-Rad Turbo Trans-Blot system. The membranes were blocked in 5% fat-free milk (Blotting grade blocker, Bio-Rad) in TBS buffer, incubated with a 1:250 dilution of the anti-AAV VP1/VP2/VP3 mouse monoclonal, B1 (61058 from Progen), or with a 1:100 of the anti-AAV2 replicase mouse monoclonal, 259.5 (61071 from Progen), or with a 1:1000 of the anti-HA tag antibody mouse monoclonal (ab18181 from Abcam) followed by a 1:5000 dilution of a m-IgGK BP-HRP antibody (sc-516102 from Santa Cruz Biotechnology). Staining was developed using Western Clarity ECL revelation kit (170-5060, Bio-Rad).

D. Quantification of AAV Particles in Hairy Roots from Brassica rapa rapa

1. rAAV2/8 Vector Produced in Hairy Roots from Brassica rapa rapa

The hairy root clones of Brassica rapa rapa were developed as previously described. Detection of assembled AAV particles was carried out in protein extracts from clone C1-38 (comprising construct no 1 as described above) and from clones C2-8 and C2-53 comprising construct no 2 as described above). A wild type clone (i.e without transgene) was included as negative control.

The clones were maintained for 20 days. Briefly, 1 g of fresh hairy roots was seeded in 100 mL of Gamborg B5 medium with 30 g/L of sucrose and incubated for 10 days at 23° C. and 100 rpm. After 10 days of growth, hairy roots were transferred into 100 mL of fresh Gamborg B5 medium with 30 g/L of sucrose, supplemented with 1 mg/L of 2,4D, and incubated for 10 more days at 23° C. and 100 rpm. At the end of the culture 100 mg of fresh biomass were collected and frozen in liquid nitrogen and stored at −80° C. for subsequent protein extraction.

On each generated sample, 3 different extraction methods (Methods B, C and D below) were tested and applied as described in table 1.

TABLE 1
description of the three extraction methods used

| Method B | Method C | Method D |
|---|---|---|
| Extraction buffer B: | Extraction buffer C: | Extraction buffer D: |
| 20 mM Tris-HCl pH 7.5 | PBS pH 7.2 | 50 mM Tris-acetate pH 7.5 |
| 150 mM NaCl | 50 mM sodium ascorbate | 1 mM EDTA |
| 10 mM MgCl₂ | 2 mM EDTA | 1% sodium ascorbate |
| 1% Triton X-100 | 1 mM PMSF | 0.5M sucrose |
| | 0.2% Triton X-100 | |
| Steps : | Steps : | Steps : |
| 100 mg of HR in 350 μL | 100 mg of HR in 350 μL | 100 mg of HR in 350 μL |
| of extraction buffer B | of extraction buffer C | of cold (4° C.) extraction |
| Grinding in Tissue Lyser | Grinding in Tissue Lyser | buffer D |
| 2 × 2 min at 25 Hz | 2 × 2 min at 25 Hz | Grinding in Tissue Lyser |
| Incubation 10 min at | Incubation 10 min at | 2 × 2 min at 25 Hz |
| room temperature | room temperature | Tissue debris removal by |
| Tissue debris removal by | Tissue debris removal by | centrifugation 10000 g, |
| centrifugation 11000 g, | centrifugation 11000 g, | 20 min, 4° C. |
| 2 min, 4° C. | 2 min, 4° C. | Centrifugation of the |
| Centrifugation of the | Centrifugation of the | supernatant 5000 g, |
| supernatant 5000 g, | supernatant 5000 g, | 5 min, 4° C. |
| 5 min, 4° C. | 5 min, 4° C. | |
| Addition of benzonase | | |
| (20 U/mL) in the | | |
| supernatant | | |
| Incubation 1 h at 37° C. | | |

The concentration in assembled viral particles (capsids/mL) was then determined by using the Progen AAV8 Capsid ELISA Kit described below.

2. rAAV8-eGFP Control Vector Preparations

A rAAV8-eGFP vector produced from mammalian cells was used as a positive control in each experiment.

The control vector was manufactured as follows. Briefly, HEK293T cells adapted to grow in suspension culture were seeded in Shake Flask (1 L) in 400 mL of F17 chemically defined culture medium. Cells were transfected with three plasmids with the PTG1+ transfection agent. Cells were collected 72H post-transfection and lysed by sonication. AAV8 particles in the culture supernatant were polyethylene glycol (PEG)-precipitated, then purified by double CsCl density gradient ultracentrifugation, and finally formulated in 1×DPBS containing Ca2+ and Mg2+ through dialysis in Slide-A-Lyzer™ 10 K MWCO cassettes (Thermo Scientific, Illkirch, France).

The concentration in viral genome/mL (VG/mL) was determined from deoxyribonuclease-resistant particles by a TaqMan real-time PCR assay. The concentration in viral particles (capsids/mL) was determined by using the Progen AAV8 Capsid ELISA Kit described below.

5 μL of control vector (which correspond to 5×10⁷ capsids) was either spiked in 100 μL of extraction buffers (B, C or D) or in 100 μL of hairy roots protein extracts obtained from a wild type clone (i.e. without transgene) according to extraction methods described above (B, C or D).

3. rAAV8-eGFP Denatured Vector Preparations

The control vector was heat-denatured at 90° C. for 15 minutes in order to serve as negative control. This way, we ensure that only assembled capsids lead to a signal in ELISA.

5 μL (which correspond to 5×10⁷ capsids) of denatured vector was either spiked in 100 μL of extraction buffers (B, C or D) or spiked in 100 μL of hairy roots protein extracts from a wild type clone (i.e without transgene).

4. AAV8 Capsid Titration by ELISA

Titration of AAV8 assembled capsids by ELISA was performed by using the Progen AAV8 Capsid ELISA Kit (PROGEN Biotechnik, Heidelberg, Germany), following the manufacturer's instructions.

### Results

The objective was to determine whether hairy roots from Brassica rapa are able to produce all proteins required to form AAV (REP, CAP and AAP proteins).

To reach this objective, molecular constructs as described above were designed, cloned and introduced in Brassica rapa using Rhizobium rhizogenes. Transgenic hairy roots of Brassica rapa rapa were generated and analyzed. The results are presented thereafter with respect to construct 1 and construct 2.

1—Analysis of the Production of AAV Proteins from Hairy Roots Containing Construct 1

Western blot analysis was performed on the protein extracts from hairy root clones containing the construct 1 has detailed above.

The Western blot analysis demonstrated the ability of the hairy roots to produce VP1, VP2 and VP3 proteins.

The Western blot analysis also demonstrated the ability of the hairy roots to produce the Rep proteins.

2—Analysis of the Production of AAV Proteins from Hairy Roots Containing Construct 2

Western blot analysis was performed on the protein extracts from hairy root clones containing the construct 2 has detailed above. The construct 2 was designed to test the ability of Brassica rapa to express VP1, VP2, VP3 and AAP (from AAV8) using different promoters: the “2*35S” promoter or the NOS promoter.

The Western blot analysis demonstrated the ability of the hairy roots to produce VP1, VP2 and VP3 proteins.

The Western blot analysis also demonstrated the ability of the hairy roots to produce the AAP-HA protein.

3. Quantification of AAV Particles in Hairy Roots from Brassica rapa rapa

We have thus demonstrated the ability of hairy roots to produce AAV Rep, VP1, VP2, VP3 and AAP proteins. The objective was then to determine whether hairy roots from Brassica rapa are able to produced assembled capsids.

The quantification of AAV particles present in hairy roots protein extracts prepared with 3 extraction methods (described in the Materials and Methods) was determined by ELISA from clones which were shown to produce VP and/or AAP proteins by Western Blot (C1-38/C2-8/C2-53).

A wild type clone (i.e without transgene) was included as negative control.

The results detailed in table 2 below demonstrate that hairy roots from Brassica rapa are able to produce assembled capsids.

TABLE 2
AAV8 titration ELISA in hairy roots protein extracts from clone C1-38, C2-8, C2-53.
A rAAV8_eGFP control vector was spiked either in extraction buffers B, C, D
or in hairy roots protein extracts from a wild type clone (i.e. without transgene)
prepared with buffer B, C, D, to serve as positive control. The same rAAV8_eGFP
vector preparation was heat denatured at 90° C. during 15 minutes and spiked
in same way either in extraction buffers B, C, D or in hairy roots protein
extracts from a wild type clone (i.e. without transgene).

| | Assay | | | |
|---|---|---|---|---|
| | buffer | Lysis buffers | | |
| | ASSBIX | B | C | D |
| Spiked rAAV8_eGFP control vector in | 1.98E+08 | 1.64E+08 | 1.85E+08 | 1.03E+08 |
| buffers (capsids/mL) | | | | |
| Heat-denatured rAAV2/8-eGFP control | NA | NA | NA | ND |
| vector (capsids/mL) | | | | |
| Spiked rAAV2/8_eGFP control vector in | NA | 1.93E+08 | 1.81E+08 | 1.51E+08 |
| protein extracts from wild type clone | | | | |
| (capsids/mL) | | | | |
| Hairy roots protein extracts clone C1-38 | NA | 8.24E+08 | 1.36E+09 | 4.21E+08 |
| (capsids/mL) | | | | |
| Hairy roots protein extracts clone C2-8 | NA | 1.79E+08 | 2.20E+09 | 1.68E+08 |
| (capsids/mL) | | | | |
| Hairy roots protein extracts clone C2-53 | NA | 4.04E+08 | ND | 2.70E+08 |
| (capsids/mL) | | | | |

NA: not applicable ; ND: not determined.

It can be noted that the titers obtained with the control rAAV8-eGFP vector spiked in either (1) extraction buffers or (2) in protein extracts from a wild type hairy root clone, are almost identical, thus validating the titration method.

The titers (capsids/mL) obtained in protein extracts from clone C1-38 (which refers to construct no 1), and from clones C2-8 and C2-53 (which refer to construct no 2) are high and comparable to the titers obtained with the control rAAV8-eGFP vector.

Titers obtained in protein extracts of hairy root clones are unexpectedly high, in view that no concentration step was carried out and that a very small amount of starting material was used for extraction and quantification.

In addition, said results showed detection of AAV assembled capsids following three different extraction methods (B, C, D), thus demonstrating the reproducibility of the method.

### REFERENCES

An, G., Costa, M. A., Mitra, A., Ha, S.-B., and Marton, L. (1988). Organ-Specific and Developmental Regulation of the Nopaline Synthase Promoter in Transgenic Tobacco Plants. PLANT PHYSIOLOGY 88, 547-552.
Bahramnejad B, Naji M, Bose R, Jha S (2019) A critical review on use of Agrobacterium rhizogenes and their associated binary vectors for plant transformation. Biotechnol Adv 50734-9750(19):30095.
Balakrishnan, B., and Jayandharan, G. R. (2014). Basic biology of adeno-associated virus (AAV) vectors used in gene therapy. Curr Gene Ther 14, 86-100.
Barajas D., Aponte-Ubillus J. J., Akeefe H., Cinek T., Peltier J., Gold D. (2017) Generation of infectious recombinant Adeno-associated virus in Saccharomyces cerevisiae. PLoS ONE. 12:e0173010.
Bartel M., Schaffer D., Büning H. (2011) Enhancing the Clinical Potential of AAV Vectors by Capsid Engineering to Evade Pre-Existing Immunity. Front. Microbiol. 2:204.
Bulgakov V P, Aminin D L, Shkryl Y N, Gorpenchenko T Y, Veremeichik G N, Dmitrenok P S, Zhuravlev Y N. (2008) Suppression of reactive oxygen species and enhanced stress tolerance in Rubia cordifolia cells expressing the rolC oncogene. Mol Plant Microbe Interact 21: 1561-1570
Christensen, A. H., Sharrock, R. A., and Quail, P. H. (1992). Maize polyubiquitin genes: structure, thermal perturbation of expression and transcript splicing, and promoter activity following transfer to protoplasts by electroporation. Plant Mol. Biol. 18, 675-689.
De Veylder, L., Van Montagu, M., and Inzé, D. (1997). Herbicide safener-inducible gene expression in Arabidopsis thaliana. Plant Cell Physiol. 38, 568-577.
Dey, N., and Maiti, I. B. (1999). Structure and promoter/leader deletion analysis of mirabilis mosaic virus (MMV) full-length transcript promoter in transgenic plants. Plant Mol. Biol. 40, 771-782.
Fei, H., Chaillou, S., Hirel, B., Mahon, J. D., and Vessey, J. K. (2003). Overexpression of a soybean cytosolic glutamine synthetase gene linked to organ-specific promoters in pea plants grown in different concentrations of nitrate. Planta 216, 467-474.
Felenbok, B., Sequeval, D., Mathieu, M., Sibley, S., Gwynne, D. I., and Davies, R. W. (1988). The ethanol regulon in Aspergillus nidulans: characterization and sequence of the positive regulatory gene alcR. Gene 73, 385-396.
Frey, A. D., Rimann, M., Bailey, J. E., Kallio, P. T., Thompson, C. J., and Fussenegger, M. (2001). Novel pristinamycin-responsive expression systems for plant cells. Biotechnol. Bioeng. 74, 154-163.
Kay, R., Chan, A., Daly, M., and McPherson, J. (1987). Duplication of CaMV 35S Promoter Sequences Creates a Strong Enhancer for Plant Genes. Science 236, 1299-1302.
Langridge, W. H., Fitzgerald, K. J., Koncz, C., Schell, J., and Szalay, A. A. (1989). Dual promoter of Agrobacterium tumefaciens mannopine synthase genes is regulated by plant growth hormones. Proc. Natl. Acad. Sci. U.S.A. 86, 3219-3223.
Ling, C., Li, B., Ma, W., Srivastava, A. (2016). Development of Optimized AAV Serotype Vectors for High-Efficiency Transduction at Further Reduced Doses. Hum Gene Ther Methods. August; 27(4):143-9.
McCarty D M, et al. (2003) Adeno-associated virus terminal repeat (TR) mutant generates self-complementary vectors to overcome the rate-limiting step to transduction in vivo. Gene Ther. 10:2112-2118.
McElroy, D., Zhang, W., Cao, J., and Wu, R. (1990). Isolation of an efficient actin promoter for use in rice transformation. Plant Cell 2, 163-171.
Odell, J. T., Nagy, F., and Chua, N. H. (1985). Identification of DNA sequences required for activity of the cauliflower mosaic virus 35S promoter. Nature 313, 810-812.
Robert, M.-A., Chahal, P. S., Audy, A., Kamen, A., Gilbert, R., and Gaillet, B. (2017). Manufacturing of recombinant adeno-associated viruses using mammalian expression platforms. Biotechnol J 12.
Rosario A M, et al. (2016). Microglia-specific targeting by novel capsid-modified AAV6 vectors. Mol Ther Methods Clin Dev. April 13; 3:16026.
Sanger, M., Daubert, S., and Goodman, R. M. (1990). Characteristics of a strong promoter from figwort mosaic virus: comparison with the analogous 35S promoter from cauliflower mosaic virus and the regulated mannopine synthase promoter. Plant Mol. Biol. 14, 433-443.
Schenk, P. M., Remans, T., Sági, L., Elliott, A. R., Dietzgen, R. G., Swennen, R., Ebert, P. R., Grof, C. P., and Manners, J. M. (2001). Promoters for pregenomic RNA of banana streak badnavirus are active for transgene expression in monocot and dicot plants. Plant Mol. Biol. 47, 399-412.
Schmulling, T., Schell, J. and Spena, A. (1988) Single genes of Agrobacterium rhizogenes influence plant development. EMBO J. 7: 2621-2629.
Schöffl, F., Rieping, M., Baumann, G., Bevan, M., and Angermüller, S. (1989). The function of plant heat shock promoter elements in the regulated expression of chimaeric genes in transgenic tobacco. MGG Molecular & General Genetics 217, 246-253.
Shirasawa-Seo, N., Sano, Y., Nakamura, S., Murakami, T, Gotoh, Y., Naito, Y., Hsia, C. N., Seo, S., Mitsuhara, I., Kosugi, S., et al. (2005). The promoter of Milk vetch dwarf virus component 8 confers effective gene expression in both dicot and monocot plants. Plant Cell Rep. 24, 155-163.
Siebertz, B., Logemann, J., Willmitzer, L., and Schell, J. (1989). cis-analysis of the wound inducible promoter wun1 in transgenic tobacco plants and histochemical localization of its expression. Plant Cell 1, 961-968.
Smith, R. H., Levy, J. R., and Kotin, R. M. (2009). A simplified baculovirus-AAV expression vector system coupled with one-step affinity purification yields high-titer rAAV stocks from insect cells. Mol. Ther. 17, 1888-1896.
Sonntag, F., Schmidt, K., and Kleinschmidt, J. A. (2010). A viral assembly factor promotes AAV2 capsid formation in the nucleolus. Proc Natl Acad Sci USA 107, 10220-10225.
Urabe, M., Nakakura, T., Xin, K.-Q., Obara, Y., Mizukami, H., Kume, A., Kotin, R. M., and Ozawa, K. (2006). Scalable generation of high-titer recombinant adeno-associated virus type 5 in insect cells. J. Virol. 80, 1874-1885.
Vercauteren, K. et al., (2016). Superior In vivo Transduction of Human Hepatocytes Using Engineered AAV3 Capsid. Mol. Ther. volume 24, issue 6, p1042-1049.
Verdaguer, B., de Kochko, A., Beachy, R. N., and Fauquet, C. (1996). Isolation and expression in transgenic tobacco and rice plants, of the cassava vein mosaic virus (CVMV) promoter. Plant Mol. Biol. 31, 1129-1139.
Weitzman, M. D., and Linden, R. M. (2011). Adeno-Associated Virus Biology. In Adeno-Associated Virus: Methods and Protocols, R. O. Snyder, and P. Moullier, eds. (Totowa, N.J.: Humana Press), pp. 1-23.
White, F. F., Garfinkel, D. J., Huffman, G. A., et al., (1983) Sequence homologous to Agrobacterium rhizogenes T-DNA in the genome of uninfected plants. Nature, vol. 301, pp. 348-350.
Xie, Y., Liu, Y., Meng, M., Chen, L., and Zhu, Z. (2003). Isolation and identification of a super strong plant promoter from cotton leaf curl Multan virus. Plant Mol. Biol. 53, 1-14.
Zhong L., et al. (2008) Next generation of adeno-associated virus 2 vectors: Point mutations in tyrosines lead to high-efficiency transduction at lower doses. Proc. Natl. Acad. Sci. U.S.A. 105, 7827-7832

1-29. (canceled)

30. A method for producing a recombinant mammalian viral vector from hairy roots of a plant comprising the steps of:
a) inducing the formation of hairy roots from said plant; and
b) transforming said plant with at least one vector containing one or more expression cassette(s); wherein the one or more expression cassette(s) comprise genes encoding the protein components required for the production of the recombinant viral vector;
wherein said plant belongs to the Brassicaceae family.

31. The method according to claim 30, wherein said plant belonging to the Brassicaceae family is selected from the group consisting of Raphanus sativus, Raphanus sativus var. niger, Brassica oleracea L. convar, Brassica napus, Arabidopsis thaliana and Brassica rapa.

32. The method according to claim 31, wherein said plant is Brassica rapa.

33. The method according to claim 30, wherein step a) is carried out by transforming the plant with a bacterial strain comprising the rol genes, wherein the bacterial strain is able to infect the plant.

34. The method according to claim 33, wherein the bacterial strain is Rhizobium rhizogenes or Agrobacterium tumefaciens.

35. The method according to claim 30, wherein the recombinant viral vector is a recombinant adeno-associated virus (AAV) viral vector.

36. The method according to claim 35, wherein the one or more expression cassette(s) comprise AAV rep and cap genes, wherein each of the AAV rep and cap genes is under the control of a promoter derived from a virus infecting Brassicaceae plants.

37. The method according to claim 36, wherein said promoter is the cauliflower mosaic virus 35S (CaMV35S) promoter.

38. The method according to claim 35, wherein the one or more expression cassette(s) comprise the genes encoding VP1, VP2, VP3, AAP (Assembly Activating Protein), Rep52 and Rep78 protein of said AAV.

39. The method according to claim 38, wherein each gene encoding VP1, VP2, VP3, AAP, Rep52 or Rep78 protein is under the control of a constitutive promoter which is selected from the cauliflower mosaic virus 35S (CaMV35S) promoter or the nopaline synthase (nos) promoter, or under the control of an inducible promoter which is the alcohol dehydrogenase (AlcA) promoter.

40. The method according to claim 38, wherein the gene encoding VP1 is under the control of the nos promoter, the gene encoding VP2 is under the control of the nos promoter, the gene encoding VP3 is under the control of the CaMV35S promoter or a functional variant thereof, and the gene encoding AAP is under the control of the CaMV35S promoter or a functional variant thereof.

41. The method according to claim 40, wherein the gene encoding VP3 is under the control of a functional variant of CaMV35S promoter having at least 60% identity to the nucleotide sequence of SEQ ID NO: 13 and the gene encoding AAP is under the control of a functional variant of CaMV35S promoter having at least 60% identity to the nucleotide sequence of SEQ ID NO: 13.

42. The method according to claim 38, wherein each gene encoding VP1, VP2, VP3 and AAP is under the control of an AlcA promoter.

43. The method according to claim 38, wherein the gene encoding VP3 is further under the control of the Tobacco Mosaic Virus Omega (TMVΩ) enhancer.

44. The method according to claim 38, wherein each gene encoding Rep52 and Rep78 is under the control of an AlcA promoter.

45. The method according to claim 38, wherein the gene encoding Rep52 is further under the control of the Tobacco Mosaic Virus Omega (TMVΩ) enhancer.

46. The method according to claim 38, wherein the gene encoding VP1, VP2, VP3, AAP, Rep52 and/or Rep78 protein is codon optimized.

47. The method according to claim 35, wherein the plant is further transformed with a vector coding for the adenoviral helper functions.

48. The method according to claim 35, wherein the plant is further transformed with a vector that comprises a viral genome comprising a gene encoding a product of interest or said vector comprises a gene encoding a product of interest flanked by two AAV-ITR sequences.

49. A hairy root culture obtainable by:
a) inducing the formation of hairy roots from a plant; and
b) transforming said plant with at least one vector containing one or more expression cassette(s); wherein the one or more expression cassette(s) comprise genes encoding the protein components required for the production of a recombinant mammalian viral vector;
wherein said plant belongs to the Brassicaceae family.

50. A recombinant mammalian viral vector obtainable by the method of claim 30.

51. A transgenic plant transformed with at least one vector containing one or more expression cassette(s); wherein the one or more expression cassette(s) comprise genes encoding the protein components required for the production of a recombinant mammalian viral vector and wherein said plant belongs to the Brassicaceae family.


SEQUENCE LISTING (23 sequences)

SEQ ID NO: 1 - DNA, 2217 bp, artificial
  cap
acggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaaagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccatagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtatcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtccccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 2 - DNA, 1932 bp, artificial
  rep
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctga

SEQ ID NO: 3 - DNA, 2217 bp, artificial
  vp1 opt
atggcagctgacggctatctacctgactggctcgaggacaatctctccgagggaattcgtgagtggtgggcccttaaacctggtgctccgaaacctaaagctaatcaacagaaacaagacgacggacggggccttgtgctacctggatacaaatacctcggtcctttcaatgggctcgacaaaggtgagcctgttaatgccgccgacgcagccgctctcgagcatgacaaagcttacgaccaacaacttcaagccggcgacaatccgtaccttcggtataatcatgctgacgctgagttccaagagcgtcttcaggaagacacgtccttcggtggaaatctcggtcgtgcagttttccaagctaaaaaacgggtcctcgaacctctcggccttgtcgaggaaggagcaaaaacggcacctggtaaaaaaagaccggtagagccttcacctcaacgttcccctgactcttccacgggaattggaaaaaaaggacagcaacctgctagaaaaagactcaatttcggccaaacaggagactcagagtcagtccctgaccctcagcctctcggtgaacctcctgcagccccttccggcgtgggtcctaatacaatggcagcaggaggcggagcacctatggcagacaataatgaaggagctgacggtgtgggcagttcttcgggtaattggcattgcgactctacatggcttggagacagagttattactactagcactcgtacttgggctcttcctacttacaataatcatctctacaaacagatttctaatggtacatcgggtggtgctactaatgacaatacttacttcggatacagcactccttggggttatttcgacttcaatagattccattgccatttctcacctcgtgactggcaacgtcttattaataataattggggtttccggcctaaaagactcagcttcaaactcttcaatattcaagttaaagaggttacgcaaaatgaaggaactaaaactattgctaataatctcactagcactattcaagtgttcacggactcggagtaccaacttccgtacgtcctcggatccgctcatcaaggatgccttcctccgttcccggccgacgtgttcatgattcctcaatacggataccttacactcaataatggcagtcaagctgtgggtcgttcttctttctactgccttgaatacttcccttcgcaaatgcttagaactggaaataatttccaattcacatacactttcgaggacgtgcctttccatagcagctacgctcatagccaaagcttggaccggcttatgaatcctcttattgaccaatacctttactacttgtcccggacacagacaacaggtggaacggcaaatacgcaaacacttggattcagccagggcggtcctaatacaatggctaatcaagcaaaaaattggcttcctggtccttgctaccgtcagcagcgtgtttcaacgacaactggtcagaataataatagcaatttcgcttggacagcaggtactaaataccatcttaatggtagaaattcattggcaaatcctggaattgcaatggcaacacataaagacgacgaggagcgtttcttccctagtaatggtattcttattttcggaaaacagaatgcagctagagacaatgccgactacagcgacgttatgctcactagcgaggaagaaattaaaactacaaatcctgtggcaacagaggaatacggcattgtggcagacaatttgcaacaacagaatacggcacctcagattggtacagttaatagccaaggtgctttacctggcatggtttggcaaaatcgggacgtgtaccttcaaggccctatttgggctaaaattcctcatacggacggaaatttccatccgtccccgcttatgggaggattcggacttaaacatcctccgcctcaaattcttattaaaaatacgcctgtacctgccgaccctccgactactttcaatcaatcaaaacttaattccttcattacgcagtacagcactggtcaagttagcgtggaaattgaatgggagcttcaaaaagaaaatagcaaacgttggaatcctgagattcaatacacttctaattactacaaatccacaagtgtggacttcgcagtcaatacagaaggagtgtactccgaacctcgtcctattggaactcgttacctcactcgtaatctttaa

SEQ ID NO: 4 - DNA, 1806 bp, artificial
  vp2 opt
atggcacctggtaaaaaaagaccggtagagccttcacctcaacgttcccctgactcttccacgggaattggaaaaaaaggacagcaacctgctagaaaaagactcaatttcggccaaacaggagactcagagtcagtccctgaccctcagcctctcggtgaacctcctgcagccccttccggcgtgggtcctaatacaatggcagcaggaggcggagcacctatggcagacaataatgaaggagctgacggtgtgggcagttcttcgggtaattggcattgcgactctacatggcttggagacagagttattactactagcactcgtacttgggctcttcctacttacaataatcatctctacaaacagatttctaatggtacatcgggtggtgctactaatgacaatacttacttcggatacagcactccttggggttatttcgacttcaatagattccattgccatttctcacctcgtgactggcaacgtcttattaataataattggggtttccggcctaaaagactcagcttcaaactcttcaatattcaagttaaagaggttacgcaaaatgaaggaactaaaactattgctaataatctcactagcactattcaagtgttcacggactcggagtaccaacttccgtacgtcctcggatccgctcatcaaggatgccttcctccgttcccggccgacgtgttcatgattcctcaatacggataccttacactcaataatggcagtcaagctgtgggtcgttcttctttctactgccttgaatacttcccttcgcaaatgcttagaactggaaataatttccaattcacatacactttcgaggacgtgcctttccatagcagctacgctcatagccaaagcttggaccggcttatgaatcctcttattgaccaatacctttactacttgtcccggacacagacaacaggtggaacggcaaatacgcaaacacttggattcagccagggcggtcctaatacaatggctaatcaagcaaaaaattggcttcctggtccttgctaccgtcagcagcgtgtttcaacgacaactggtcagaataataatagcaatttcgcttggacagcaggtactaaataccatcttaatggtagaaattcattggcaaatcctggaattgcaatggcaacacataaagacgacgaggagcgtttcttccctagtaatggtattcttattttcggaaaacagaatgcagctagagacaatgccgactacagcgacgttatgctcactagcgaggaagaaattaaaactacaaatcctgtggcaacagaggaatacggcattgtggcagacaatttgcaacaacagaatacggcacctcagattggtacagttaatagccaaggtgctttacctggcatggtttggcaaaatcgggacgtgtaccttcaaggccctatttgggctaaaattcctcatacggacggaaatttccatccgtccccgcttatgggaggattcggacttaaacatcctccgcctcaaattcttattaaaaatacgcctgtacctgccgaccctccgactactttcaatcaatcaaaacttaattccttcattacgcagtacagcactggtcaagttagcgtggaaattgaatgggagcttcaaaaagaaaatagcaaacgttggaatcctgagattcaatacacttctaattactacaaatccacaagtgtggacttcgcagtcaatacagaaggagtgtactccgaacctcgtcctattggaactcgttacctcactcgtaatctttaa

SEQ ID NO: 5 - DNA, 1608 bp, artificial
  vp3 opt
atggcagcaggaggcggagcacctatggcagacaataatgaaggagctgacggtgtgggcagttcttcgggtaattggcattgcgactctacatggcttggagacagagttattactactagcactcgtacttgggctcttcctacttacaataatcatctctacaaacagatttctaatggtacatcgggtggtgctactaatgacaatacttacttcggatacagcactccttggggttatttcgacttcaatagattccattgccatttctcacctcgtgactggcaacgtcttattaataataattggggtttccggcctaaaagactcagcttcaaactcttcaatattcaagttaaagaggttacgcaaaatgaaggaactaaaactattgctaataatctcactagcactattcaagtgttcacggactcggagtaccaacttccgtacgtcctcggatccgctcatcaaggatgccttcctccgttcccggccgacgtgttcatgattcctcaatacggataccttacactcaataatggcagtcaagctgtgggtcgttcttctttctactgccttgaatacttcccttcgcaaatgcttagaactggaaataatttccaattcacatacactttcgaggacgtgcctttccatagcagctacgctcatagccaaagcttggaccggcttatgaatcctcttattgaccaatacctttactacttgtcccggacacagacaacaggtggaacggcaaatacgcaaacacttggattcagccagggcggtcctaatacaatggctaatcaagcaaaaaattggcttcctggtccttgctaccgtcagcagcgtgtttcaacgacaactggtcagaataataatagcaatttcgcttggacagcaggtactaaataccatcttaatggtagaaattcattggcaaatcctggaattgcaatggcaacacataaagacgacgaggagcgtttcttccctagtaatggtattcttattttcggaaaacagaatgcagctagagacaatgccgactacagcgacgttatgctcactagcgaggaagaaattaaaactacaaatcctgtggcaacagaggaatacggcattgtggcagacaatttgcaacaacagaatacggcacctcagattggtacagttaatagccaaggtgctttacctggcatggtttggcaaaatcgggacgtgtaccttcaaggccctatttgggctaaaattcctcatacggacggaaatttccatccgtccccgcttatgggaggattcggacttaaacatcctccgcctcaaattcttattaaaaatacgcctgtacctgccgaccctccgactactttcaatcaatcaaaacttaattccttcattacgcagtacagcactggtcaagttagcgtggaaattgaatgggagcttcaaaaagaaaatagcaaacgttggaatcctgagattcaatacacttctaattactacaaatccacaagtgtggacttcgcagtcaatacagaaggagtgtactccgaacctcgtcctattggaactcgttacctcactcgtaatctttaa

SEQ ID NO: 6 - DNA, 594 bp, artificial
  AAP opt
atggccacacaaagtcaattccaaactctcaatctctcggagaatctccaacaacgtcctcttgtgtgggacctgatacagtggcttcaagccgtggcccatcagtggcaaacaataacgaaagccccgacggagtgggtagtccctcgggaaattggaattgccattcctcatggatgggccacagagtcatcacctcctgcacctgaacctggtccttgccctcctacaacaactacttccacaagcaaatcccctacgggtcatcgggaggagcctcctacgacaacacctacatcggcaacagcacctcctggtggcattttgacattaacagactctacagctacattccatcatgtgacaggaagcgactcatcaacaacaacaggtgactctggacctagagactcagcatcaagctcctcaacatctaggtcaaggaggtcacgtagaatgaaagcacctagaccttcgcctataacttcacctgcaccttctaggtgcttacggacacggagtactagctgccgtacgttctcggcacttcctactagggcagcttgcctccgttctcggcggacgtgctcatga

SEQ ID NO: 7 - DNA, 1194 bp, artificial
  rep52 opt
atggagcttgttggttggctcgtggacaaaggtattacttcggagaaacaatggattcaagaggaccaagcttcatacatttctttcaatgccgcttctaattcgcggtcgcagattaaagcagctttggacaatgccggtaaaattatgagccttacaaaaactgctcctgactaccttgtgggacaacaacctgtggaggacatttctagcaatcggatttataaaattttggaactgaatggttacgaccctcagtatgccgcatctgttttccttggttgggctacgaaaaaattcggaaaaaggaatactatttggcttttcggtcctgcaacaactggtaaaactaatattgccgaggctatagctcatacagtgcctttctacggttgcgtaaattggactaatgagaatttccctttcaatgactgcgttgacaaaatggtgatttggtgggaggagggtaaaatgactgctaaagttgtggagtcggctaaagctattctcggtggtagcaaagtgcgtgtggaccaaaaatgcaaatcttcggctcaaatagacccgacacctgtgattgttacttctaatactaatatgtgcgctgtgattgacggtaattcaacgactttcgaacatcaacaaccgttgcaggaccggatgttcaaattcgaactcactcgtcgtcttgaccatgacttcggtaaagttactaaacaagaagttaaagacttcttccggtgggcaaaagaccatgtggtcgaggtggagcatgaattctacgttaaaaaaggcggtgctaaaaaaagacctgctcctagtgacgcagacataagtgagcctaaacgggtgcgtgagtcagtcgcccaaccttcgacgtcagacgccgaagcatcgattaattacgcagacaggtaccagaataaatgctcccgtcatgtgggaatgaatcttatgcttttcccttgcagacagtgcgagagaatgaatcaaaattcaaatatttgcttcacacatggtcaaaaagactgcttagagtgcttccctgtgtcagaatcccagcctgtctccgttgttaaaaaagcctatcaaaaactttgctacattcatcatattatgggtaaagtgcctgacgcatgcacagcttgcgaccttgttaatgtggacttggacgactgcattttcgaacagtaa

SEQ ID NO: 8 - DNA, 1866 bp, artificial
  rep78 opt
atgccgggtttctacgagattgtgattaaagttcctagcgacctagacgagcatcttcctggaatttccgacagcttcgtgaattgggtggctgagaaagaatgggagttgccgcctgactccgacatggaccttaatcttattgagcaagcacctcttactgtggctgagaaacttcaacgtgacttccttacggaatggcgtcgtgtgagtaaagctccggaggcactattcttcgtgcagttcgagaaaggtgagagctacttccatatgcatgtgctcgtggaaactactggtgtgaaatctatggtcttgggtcgtttccttagtcaaattcgtgaaaaacttattcaaagaatttaccgtggtattgagccgacattgcctaattggttcgccgttacaaaaactagaaatggagctggtggaggtaataaagtggtggacgagtgctacattcctaattacttgctccctaaaactcaacctgagctccaatgggcctggacaaatatggaacaatatttaagcgcttgcttgaatctcacggagcgtaaacggttggtggcccaacatcttacgcatgtgtcgcaaacgcaagagcaaaataaagagaatcaaaatcctaattccgacgccccggtgattagatcaaaaacatcagctaggtacatggagcttgttggttggctcgtggacaaaggtattacttcggagaaacaatggattcaagaggaccaagcttcatacatttctttcaatgccgcttctaattcgcggtcgcagattaaagcagctttggacaatgccggtaaaattatgagccttacaaaaactgctcctgactaccttgtgggacaacaacctgtggaggacatttctagcaatcggatttataaaattttggaactgaatggttacgaccctcagtatgccgcatctgttttccttggttgggctacgaaaaaattcggaaaaaggaatactatttggcttttcggtcctgcaacaactggtaaaactaatattgccgaggctatagctcatacagtgcctttctacggttgcgtaaattggactaatgagaatttccctttcaatgactgcgttgacaaaatggtgatttggtgggaggagggtaaaatgactgctaaagttgtggagtcggctaaagctattctcggtggtagcaaagtgcgtgtggaccaaaaatgcaaatcttcggctcaaatagacccgacacctgtgattgttacttctaatactaatatgtgcgctgtgattgacggtaattcaacgactttcgaacatcaacaaccgttgcaggaccggatgttcaaattcgaactcactcgtcgtcttgaccatgacttcggtaaagttactaaacaagaagttaaagacttcttccggtgggcaaaagaccatgtggtcgaggtggagcatgaattctacgttaaaaaaggcggtgctaaaaaaagacctgctcctagtgacgcagacataagtgagcctaaacgggtgcgtgagtcagtcgcccaaccttcgacgtcagacgccgaagcatcgattaattacgcagacaggtaccagaataaatgctcccgtcatgtgggaatgaatcttatgcttttcccttgcagacagtgcgagagaatgaatcaaaattcaaatatttgcttcacacatggtcaaaaagactgcttagagtgcttccctgtgtcagaatcccagcctgtctccgttgttaaaaaagcctatcaaaaactttgctacattcatcatattatgggtaaagtgcctgacgcatgcacagcttgcgaccttgttaatgtggacttggacgactgcattttcgaacagtaa

SEQ ID NO: 9 - DNA, 1027 bp, artificial
  p35S
actagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcga

SEQ ID NO: 10 - DNA, 226 bp, artificial
  t35S
cggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacct

SEQ ID NO: 11 - DNA, 291 bp, artificial
  pnos
atacatgagaattaagggagtcacggtatgacccccgccgatgacgcgggacaagccgttttacgtttggaactgacagaaccgcaacgttgaaggagccactgagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggc

SEQ ID NO: 12 - DNA, 496 bp, artificial
  tnos
gcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcg

SEQ ID NO: 13 - DNA, 1329 bp, artificial
  2*35S
actagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaatgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcga

SEQ ID NO: 14 - DNA, 246 bp, artificial
  pAlca
cgggatagttccgacctaggattggatgcatgcggaaccgcacgagggcggggcggaaattgacacaccactcctctccacgcaccgttcaagaggtacgcgtatagagccgtatagagcagagatggagcactttctggtactgtccgcacgggatgtccgcacggagagccacaaacgagcggggccccgtacgtgctctcctaccccaggatcgcatccccgcatagctgaacatctatataa

SEQ ID NO: 15 - DNA, 2466 bp, artificial
  AlcR opt
atggcagatacgcgccgacgccagaatcatagctgcgatccctgtcgcaagggcaagcgacgctgtgatgccccggaaaatagaaacgaggccaatgaaaacggctgggtttcgtgttcaaattgcaagcgttggaacaaggattgtaccttcaattggctctcatcccaacgctccaaggcaaaaggggctgcacctagagcgagaacaaagaaagccaggaccgcaacaaccaccagtgaaccatcaacttcagctgcaacaatccctacaccggaaagtgacaatcacgatgcgcctccagtcataaactctcacgacgcgctcccgagctggactcaggggctactctcccaccccggcgaccttttcgatttcagccactctgctattcccgcaaatgcagaagatgcggccaacgtgcagtcagacgcaccttttccgtgggatctagccatccccggtgatttcagcatgggccaacagctcgagaaacctctcagtccgctcagttttcaagcagtccttcttccgccccatagcccgaacacggatgacctcattcgcgagctggaagagcagactacggatccggactcggttaccgatactaatagtgtacaacaggtcgctcaagatggatcgctatggtctgatcggcagtcgccgctactgcctgagaacagtctgtgcatggcctcagacagcacagcacggcgatatgcccgttccacaatgacgaagaatctgatgcgaatctaccacgatagtatggagaatgcactgtcctgctggctgacagagcacaattgtccatactccgaccagatcagctacctgccgcccaagcagcgggcggaatggggcccgaactggtcaaacaggatgtgcatccgggtgtgccggctagatcgcgtatctacctcattacgcgggcgcgccctgagtgcggaagaggacaaagccgcagcccgagccctgcatctggcgatcgtagcttttgcgtcgcaatggacgcagcatgcgcagaggggggctgggctaaatgttcctgcagacatagccgccgatgagaggtccatccggaggaacgcctggaatgaagcacgccatgccttgcagcacacgacagggattccatcattccgggttatatttgcgaatatcatcttttctctcacgcagagtgtgctggatgatgatgagcagcacggtatgggtgcacgtctagacaagctactcgaaaatgacggtgcgcccgtgttcctggaaaccgcgaaccgtcagctttatacattccgacataagtttgcacgaatgcaacgccgcggtaaggctttcaacaggctcccgggaggatctgtcgcatcgacattcgccggtattttcgagacaccgacgccgtcgtctgaaagcccacagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccaccaaggcgcggcgctgaaacgccgatcaacctagactgctgggagcccccgagacaggtcccgagcaatcaagaaaagagcgacgtatggggcgacctcttcctccgcacctcggactctctcccagatcacgaatcccacacacaaatctctcagccagcggctcgatggccctgcacctacgaacaggccgccgccgctctctcctctgcaacgcccgtcaaagtcctcctctaccgccgcgtcacgcagctccaaaccctcctctatcgcggcgccagccctgcccgccttgaagcggccatccagagaacgctctacgtttataatcactggacagcgaagtaccaaccatttatgcaggactgcgttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtaacaaagctaaggctcgataatgcactagcagttagtgcccttgcgcgctcttcactccgaggccaggagctggacccgggcaaagcatctccgatgtatcgccatttccatgattctctgaccgaggtggcattcctggtagaaccgtggaccgtcgttcttattcactcgtttgccaaagctgcgtatatcttgctggactgtttagatctggacggccaaggaaatgcactagcggggtacctgcagctgcggcaaaattgcaactactgcattcgggcgctgcaatttctgggcaggaagtcggatatggcggcgctggttgcgaaggatttagagagaggtttgaatgggaaagttgacagctttttgtag

SEQ ID NO: 16 - DNA, 66 bp, artificial
  tmV omega
atttttacaacaattaccaacaacaacaaacaacaaacaacattacaattactatttacaattaca

SEQ ID NO: 17 - protein, 738 aa, artificial
  VP1
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 18 - protein, 601 aa, artificial
  VP2
MAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 19 - protein, 535 aa, artificial
  VP3
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 20 - protein, 197 aa, artificial
  AAP
MATQSQFQTLNLSENLQQRPLVWDLIQWLQAVAHQWQTITKAPTEWVVPREIGIAIPHGWATESSPPAPEPGPCPPTTTTSTSKSPTGHREEPPTTTPTSATAPPGGILTLTDSTATFHHVTGSDSSTTTGDSGPRDSASSSSTSRSRRSRRMKAPRPSPITSPAPSRCLRTRSTSCRTFSALPTRAACLRSRRTCS

SEQ ID NO: 21 - protein, 397 aa, artificial
  Rep52
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 22 - protein, 621 aa, artificial
  Rep78
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 23 - protein, 821 aa, artificial
  ALCR
MADTRRRQNHSCDPCRKGKRRCDAPENRNEANENGWVSCSNCKRWNKDCTFNWLSSQRSKAKGAAPRARTKKARTATTTSEPSTSAATIPTPESDNHDAPPVINSHDALPSWTQGLLSHPGDLFDFSHSAIPANAEDAANVQSDAPFPWDLAIPGDFSMGQQLEKPLSPLSFQAVLLPPHSPNTDDLIRELEEQTTDPDSVTDTNSVQQVAQDGSLWSDRQSPLLPENSLCMASDSTARRYARSTMTKNLMRIYHDSMENALSCWLTEHNCPYSDQISYLPPKQRAEWGPNWSNRMCIRVCRLDRVSTSLRGRALSAEEDKAAARALHLAIVAFASQWTQHAQRGAGLNVPADIAADERSIRRNAWNEARHALQHTTGIPSFRVIFANIIFSLTQSVLDDDEQHGMGARLDKLLENDGAPVFLETANRQLYTFRHKFARMQRRGKAFNRLPGGSVASTFAGIFETPTPSSESPQLDPVVASEEHRSTLSLMFWLGIMFDTLSAAMYQRPLVVSDEDSQISSASPPRRGAETPINLDCWEPPRQVPSNQEKSDVWGDLFLRTSDSLPDHESHTQISQPAARWPCTYEQAAAALSSATPVKVLLYRRVTQLQTLLYRGASPARLEAAIQRTLYVYNHWTAKYQPFMQDCVANHELLPSRIQSWYVILDGHWHLAAMLLADVLESIDRDSYSDINHIDLVTKLRLDNALAVSALARSSLRGQELDPGKASPMYRHFHDSLTEVAFLVEPWTVVLIHSFAKAAYILLDCLDLDGQGNALAGYLQLRQNCNYCIRALQFLGRKSDMAALVAKDLERGLNGKVDSFL